SEQUENCE LISTING (7 sequences)

SEQ ID NO: 1 - DNA, 45 bp, Artificial Sequence
  Description of Artificial Sequence Synthetic oligonucleotide
gcgagcctgggattaaataaaatagtaagaatgtatagcgctcgc

SEQ ID NO: 2 - DNA, 34 bp, Artificial Sequence
  Description of Artificial Sequence Synthetic oligonucleotide
gcgagcgtatctagctatgttgatggtggctcgc

SEQ ID NO: 3 - DNA, 32 bp, Artificial Sequence
  Description of Artificial Sequence Synthetic oligonucleotide
gcgagcctgtacgtgtggcagttgctgctcgc

SEQ ID NO: 4 - DNA, 17 bp, Artificial Sequence
  Description of Artificial Sequence Synthetic oligonucleotide
tggttggtgtggttggt

SEQ ID NO: 5 - DNA, 11 bp, Artificial Sequence
  Description of Artificial Sequence Synthetic oligonucleotide
gataccgagcg

SEQ ID NO: 6 - DNA, 28 bp, Artificial Sequence
  Description of Artificial Sequence Synthetic oligonucleotide
cgctcggtatctagctatgttgatggtg

SEQ ID NO: 7 - DNA, 22 bp, Artificial Sequence
  Description of Artificial Sequence Synthetic oligonucleotide
gtatctagctatgttgatggtg

(19) United States
(12) Patent Application Publication
(10) Pub. No.: US 2008/0227086 A1
(43) Pub. Date: Sep. 18, 2008

(54) Test System and Method for the Detection of Analytes

(76) Inventor: Frank Vitzthum, Lahntal-Sterzhausen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/587,831

(22) PCT Filed: Jan. 31, 2005
(86) PCT No.: PCT/EP2005/000906

§ 371 Date: Jul. 28, 2006

(30) Foreign Application Priority Data
Jan. 30, 2004 (DE) 10 2004 004 882.7

(51) Int. Cl. C12Q 1/68; C12M 1/00

(52) U.S. Cl. 435/6; 435/287.2

(57) ABSTRACT

The invention relates to analytical test systems and analytical methods, in which molecular switches are used for the qualitative and quantitative determination of analytes in a sample, which have a broad application by means of a selection of the molecular switch suitable for the analyte. The molecular switch comprises a probe, preferably a nucleic acid or a nucleic acid derivative, coupled to a catalytic component, preferably an enzyme. The analyte induces a conformation change in the probe, which alters the accessibility for a substrate in the probe to catalytic components and the change in substrate turnover, corresponding to the change in catalytic activity, is measured.

The invention relates to an analytical test system and a method for specifically and sensitively detecting analytes in a sample to be investigated.

A large number of analytical methods are available for detecting and quantifying substances. In principle, it is possible, in this connection, to differentiate between methods which detect the substance to be analyzed or to be detected, i.e. the analyte, directly or indirectly. In the case of direct methods, characteristic physicochemical properties of the analyte are exploited for the purpose of detecting it as specifically and sensitively as possible. Chromatographic methods such as high performance liquid chromatography (HPLC) or gas chromatography (GC) are, for example, employed in this connection. Techniques from the fields of spectrophotometry, resonance spectroscopy, mass spectroscopy, etc., are also used (Becker, Berger et al. 1993; Hesse, Meier et al. 1995; Rehm 2002); cf. reference list at the end of the description.

In principle, the techniques which are also used in the case of direct analytical methods are employed in the case of indirect detection methods. However, in the latter instance, additional, specific binding processes or chemical reactions are exploited in order to detect analytes specifically and sensitively in accordance with their steric, chemical and physical properties. Suitable technologies are used to measure the binding processes or the chemical reactions and consequently detect analytes.

As a rule, chemical reactions are used to detect analytes in order to increase specificity and/or sensitivity. In this case, products which can as a rule be detected more sensitively than the analytes are generally generated from the analyte by way of selective reactions. Catalysts are also employed in these reactions in order to accelerate them. Because of their substrate specificity and their high catalytic efficiency, enzymes are, in particular, used for these purposes (Bergmeyer 1965; Bisswanger 1994).

Where appropriate, the analyte-specific generation of reaction products is also exploited in order to enable a particular detection technology to be applied. For example, the generation of a chromophoric reaction product having absorption properties in appropriate wavelength ranges can be of advantage for a simple and sensitive spectrophotometric detection.

If catalysts are used in chemical or biochemical reactions, the sensitivity and, where appropriate, the specificity can then be further increased by means of the following methods. In the first place, catalysts, preferably enzymes, can be used for selectively amplifying extremely low concentrations of an analyte. It is only after or during the amplification of the analyte that the latter is detected specifically and sensitively. This is the case, for example, with regard to the polymerase chain reaction (PCR) (Saiki, Scharf et al. 1985; Mullis 1987). In this reaction, thermal cycles are used to amplify particular nucleic acid sequences in the presence of a polymerase, appropriate “primers”, nucleoside triphosphates and other cofactors. In the second place, appropriate catalysts can be used to carry out a signal amplification. This is effected, in particular, by means of enzyme reactions, which transform substrates in the presence of an analyte. The specificity of these methods is achieved, for example, by means of immunological techniques or tests.

These immunological tests, i.e. enzyme immunoassays (EIAs), are used, in particular, in the life sciences sphere and in diagnostics for determining very low quantities of analytes in biological samples. The most important EIAs include enzyme-linked immunosorbent assay (ELISA) and the enzyme-multiplied immunotechnique (EMIT).

EMIT is a homogeneous liquid phase test system that is chiefly used for determining low molecular weight substances such as a variety of pharmaceuticals, hormones or metabolites. Nucleic acids are not detected using EMIT. While EMIT is as a rule more rapid than an ELISA, it does not generally achieve the sensitivity of an ELISA, either.

An ELISA is a heterogeneous solid-phase assay which is mainly used for detecting macromolecules such as antigens and antibodies. Some ELISA methods are also used for detecting nucleic acids. However, this detection is as a rule only effected indirectly by way of antigen-labeled nucleic acids. In general, an expensive and time-consuming amplification of the target sequence, for example by means of PCR using antigen-labeled primers, is firstly carried out in this case. It is only after the amplification product has hybridized to the immobilized homologous oligonucleotides, and after further binding processes, that the actual enzymatic test can be carried out.

The object of the present invention was therefore to make available an analytical test system, and a corresponding method, which can be used flexibly for a large number of different analytes and which are at the same time specific and economical.

The analytical test systems according to the invention enable the analytes to be detected rapidly, specifically, economically and in a highly sensitive manner. This invention is consequently of great help and interest for scientific and diagnostic purposes.

In addition to the rapid, specific, economical and highly sensitive detection of analytes, in particular nucleic acids, the invention furthermore provides the possibility of configuring a corresponding analytical system in a very flexible manner. According to the invention, it is possible to determine both low molecular weight and high molecular weight analytes. Furthermore, the described system can make use of a large number of catalytic systems and can be operated heterogeneously as well as homogeneously.

New molecular switch systems form the foundation for these novel analytical systems. The molecular switch systems are based on combining a catalytic component with a component which functions as a probe. In this connection, at least one catalytic component is combined with at least one probe in a manner which is such that, under selected conditions, the catalytic activity of the molecular switch system is influenced, and thereby altered, in the presence of a particular analyte. In this connection, the change in the catalytic activity can relate either to the extent of the catalytic activity or to its specificity. Detection of the altered catalytic activity can consequently be used for specifically and sensitively detecting an analyte.

The catalytic component is characterized by the fact that it converts one or more substrates into one or more products or is involved in this process. On the one hand, this means that the catalytic component itself operates as a catalyst and is consequently able to accelerate the rate of a reaction. Since the forward and backward reactions are accelerated equally, the catalyst does not alter the equilibrium position (Becker, Berger et al. 1993; Bisswanger 1994). On the other hand, the catalytic component can be involved in a catalytic process without this component having to possess direct catalytic properties.

According to the invention, any catalyst can be employed as catalytic component. Both inorganic and organic compounds which possess catalytic activity are suitable for being used as catalytic components. Inorganic and organic compounds which function as acids and/or bases are particularly suitable. Preference is given to using Lewis acids or Lewis bases. Furthermore, inorganic and organic compounds which are catalytically active and which are involved in transferring electrons, and thereby support redox reactions, are to be employed, in particular.

For example, it is possible to use organic compounds such as acidic or basic and/or electron-transferring, i.e. redox-active, aromatic compounds, heteroaromatic compounds, organic complexes, proteins, in particular enzymes, and also catalytically active antibodies or nucleic acids having catalytic activity, and their derivatives.

Examples of inorganic compounds which it is possible to use are metals, alloys, metal oxides, transition metal complexes and electrode systems. In general, metals, such as iron, cobalt, nickel, palladium, platinum, copper, silver, etc., and their alloys, salts, oxides and sulfides, organometallic compounds and transition metal complexes function as catalytic Lewis acids or Lewis bases and/or electron carriers and can consequently be employed, in accordance with the invention, as catalytic component or a constituent thereof (Becker, Berger et al. 1993).

The choice of the optimal inorganic catalyst depends on the reaction to be catalyzed. In a preferred embodiment, it is possible, for example, to use the transition metal complexes potassium hexacyanoferrate II or III in connection with catalyzing redox reactions, for example in connection with transforming redox-active substances such as phenazine methosulfate (PMS), benzoquinone, etc., but preferably, for example, a tetrazolium salt or the corresponding formazane. In this case, it is possible, for example, to use tetrazolium salts such as nitroblue tetrazolium, in particular iodonitrotetrazolium chloride.

In another preferred embodiment, inorganic compounds, in particular metals, alloys and metal oxides, are used as electrodes which are employed as a constituent of the catalytic components or as the catalytic component itself. In principle, any conductive materials can be used in this connection. For example, in addition to using metals, alloys and metal oxides, it is also possible to use conductive plastics or ceramics or other composite materials. When electrodes which are connected to an electrical potential are being used, one is not then necessarily dealing with a catalyst in the narrower sense. In this case, the electrodes are only being used for transferring charge carriers, i.e. electrons, which, for example, support a redox system, in particular a “redox cycling”. In this case, too, the choice of the optimal electrode depends on the reaction as silver, gold and platinum are, in particular, to be employed. Platinum is to be particularly preferred on account of its stability. Silver and gold are to be used, in particular, when it is a matter of using the electrode to carry out coupling reactions. Gold is preferably to be used in this connection (Hintsche 1999).

When organic compounds are being employed as catalysts, it is possible, for example, to use catalytically active nucleic acids or their derivatives as the catalytic component or as a constituent of the catalytic component. Ribonucleic acids, deoxyribonucleic acids and nucleic acid derivatives can be used in this connection. For example, it is possible to use nucleic acids containing sugar derivatives, in particular those of the pentopyranosyl-(2′-4′)-oligonucleotide family (Beier, Reck et al. 1999). In this connection, it is also possible to conceive of using riboses whose 2′ oxygen atom is linked to the 4′ carbon atom by way of a methylene bridge (“locked nucleic acids”) (Kurreck, Wyszko et al. 2002). Changes in the backbone of the catalytic nucleic acid do not have to relate only to the sugars which are used but can also relate to the way the sugars are linked to each other. It is naturally also possible to conceive of completely replacing the sugar phosphate backbone with other components. This is the case, for example, with the peptide nucleic acids (Nielsen and Egholm 1999). In addition to using nucleic acids having a derivatized backbone, it is also possible to use nucleic acids which contain unusual bases such as deaminoadenosine, inosine, etc., or biotinylated and also digoxigenized bases and other derivatives.

Apart from nucleic acids, catalytically active proteins, such as catalytically active antibodies and enzymes, are also suitable for being used as catalytic component constituents or as catalytic components themselves. In principle, it is possible to use enzymes belonging to any enzyme classes (oxidoreductases, transferases, hydrolases, lyases, isomerases and ligases) (Bergmeyer 1965). This also relates to the enzymatic activity of catalytically active antibodies or nucleic acids.

Since oxidoreductases are very suitable for enzymatic analysis in general, they are of particular interest for the application in accordance with the invention. For example, it is possible to use peroxidases, such as catalases, etc., particularly, however, horseradish peroxidases and NADH peroxidases. It is also possible to use oxidases such as cholesterol oxidases, sulfite oxidases, etc., in particular, however, xanthine oxidases, ascorbic acid oxidases, glucose oxidases, glutamate oxidases, A and B monoamine oxidases, semicarbazide-sensitive amine oxidases, choline oxidases and galactose oxidases. It is likewise possible to use reductases such as glutathione reductases. In this case, redox-active proteins such as thioredoxin, glutaredoxin, etc., may also be mentioned, in particular. The oxidoreductase luciferase may be of particular interest. Other oxidoreductases of interest, for example dehydrogenases such as formate dehydrogenases, glutamate dehydrogenases, lactate dehydrogenases, alcohol dehydrogenases, sorbitol dehydrogenases, malate dehydrogenases, malate enzymes, isocitrate dehydrogenases, galactose dehydrogenases, glucose-6-phsophate dehydrogenases, 6-phosphogluconate dehydrogenases, dihydroliponamide dehydrogenases and, in particular, the diaphorases are likewise suitable. Enzymes such as diaphorases, exhibiting a high degree of stability towards the denaturing conditions such as high temperatures, are of particular interest (Vitzthum, Bisswanger et al. 2000).

In the case of diaphorases, the Clostridium kluyveri diaphorase is in turn particularly suitable since this enzyme is a monomer. Monomers have the advantage that it is usually easier to prepare conjugates and fewer byproducts are formed.

Enzymes such as the Scyliorhinus canicula diaphorase may also be of particular interest (Vitzthum, Bisswanger et al. 2000). While the enzyme is inactive at high temperatures, e.g. the conditions for denaturing and, where appropriate, hybridizing the probes and analytes, it is not irreversibly denatured. If the temperature is reduced, the enzyme is active once again. This is advantageous if the substrates are already present at the beginning of the process since the reaction only gets going significantly when the temperature falls to a particular value. It is therefore possible to reduce a certain degree of basal activity which otherwise occurs during the denaturing and hybridization conditions. This has a positive effect on the specificity and sensitivity of the system.

Transferases which can be used are, for example, enzymes such as phosphotransacetylases, glucokinases, acetate kinases, gluconate kinases, glycerol kinases, pyruvate kinases, glutamate oxaloacetate transaminases (GOT), glutamate pyruvate transaminases (GTPs), etc., in particular, however hexokinases. Enzymes such as hexokinases offer the advantage that relatively large structural changes occur when the substrates are transformed, which means that the influence of analytes on the activity of a molecular switch using corresponding enzymes can be unexpectedly large. In the case of hexokinases, the structural change is based on what is termed an “induced fit”.

Hydrolases which can be used are, for example, enzymes such as ureases (uricase), amylases (amyloglucosidases), lactases, β-fructosidases (invertases, saccharases), maltases, β-galactosidases, maltose phosphorylase, pyrophosphatase, muramidases (lysozymes), neuramidases (sialidases), PNGaseF, endo-glycosidase (endo-alpha-N-acetylgalactosamidase) D, endoglycosidases F, endoglycosidase H, acetylcholin-esterases, collagenases, gelatinases, sphingomyelinase, etc. Lipases, in particular phospholipases (C and D phospholipases and also phosphatidylcholine-specific phospholipase C) are likewise of interest. It is also possible to use phosphatases, in particular, however, the alkaline and acid phosphatases such as the serine/threonine phosphatases (PP2A, PP1, PP-2B, etc.) and the tyrosine phosphatases (CD-45, PTP-1B, LAR, etc.). In this case, the prostatic acid phosphatase and the protein phosphatase 1 may be mentioned, in particular. It is furthermore possible to use proteases such as metalloproteases, serine proteases, acid proteases and cysteine proteases. Thermolysin, chymotrypsins, trypsins, proteinase K, caspases, elastases, papains, pepsins and cathepsins are to be used, in particular, in this case.

Lyases which can be used are, for example, the citrate lyases or citrate synthases and oxalate decarboxylase, etc.

Isomerases which can be used are, for example, the phosphoglucose isomerases and the mutarotases (aldolase 1-epimerases), etc.

Examples of relevant ligases are acetyl-CoA synthetases, NAD synthases, glutamate-cysteine ligases, homoglutathione synthases, etc.

In addition to the enzymes, it is also possible to use low molecular weight organic compounds, in particular redox-active substances, for example aromatic or heteroaromatic compounds such as benzoquinone, phenazine methosulfate (PMS), 2,6-dichlorophenolindophenol (DCPIP), methyl viologen, flavine mononucleotide (FMN), flavine adenine dinucleotide, lipoic acid, ascorbic acid, tocopherols, resorufin, resazurin, porphyrins, heme compounds, biliverdin, and bilirubin as catalysts. Nucleic acids, such as ribonucleic acids and deoxyribonucleic acids, and their derivatives, such as peptide nucleic acids or locked nucleic acids, are preferably employed as probes. In addition to using nucleic acids having a derivatized backbone, it is naturally also possible, in this case, to use nucleic acids which contain unusual bases (see above). The nucleic acid probes are preferably oligonucleotides. In principle, the probes are present in two forms, i.e. in a hybridized form and in an unhybridized form. The hybridized form can, for example, be one of the double helix structures or else a triple helix or a quadruplex structure (Rosu, Gabelica et al. 2002). The hybridized structure is based either on the intermolecular hybridization of two homologous nucleic acid single strands which are not covalently bonded to each other or on the intramolecular hybridization of a single nucleic acid strand due to the presence of homologous intramolecular regions which permit the formation of a secondary structure.

In another embodiment, the nucleic acid probe comprises or contains one or more coupling groups and/or binding components, i.e. a partner in a binding partner system. These structures can be introduced by conjugation via any component of the nucleic acid, i.e. base, backbone, or sugar or phosphate group. They can be integrated in the region of the 3′ end or the 5′ end or else in the middle region of the probe.

In principle, a distinction should be made here between coupling components and binding components or binding partner systems. Coupling components are used for combining different components and lead to permanent noncovalent or covalent bonding, i.e. to the conjugation, combination or linkage of different components. Coupling components ensure stable conjugation of components, at least under the experimental or process conditions. More precisely, the equilibrium between free and bound coupling components shifts hardly at all under the experimental or process conditions and is only to a very slight extent dependent on the experimental or process conditions. The bonds of these coupling components no longer have to be absolutely stable under extreme conditions such as very high or low pH values, very high or very low ionic strengths or very high temperatures, which conditions are usually not relevant for implementing experiments using this test system.

By contrast, under the preferred experimental or process conditions, binding partner systems exhibit a higher degree of bond reversibility. That is, in the presence of an analyte and/or in connection with a change in parameters such as temperature and/or solvent composition, an existing bond may be cut or a bond which does not exist may be rendered possible. More precisely, the equilibrium between free and bound binding partners shifts under the experimental or process conditions and depends on these conditions.

Binding partner systems are therefore suitable for reversibly binding probe components, catalytic components and analytes. These binding partner systems can be employed, in particular, when other analytes are to be detected in addition to nucleic acids.

The binding partners can be macromolecules, or particular domains of macromolecules, which exhibit an affinity towards particular ligands. For example, it is possible to use receptors, enzymes, antibodies or their binding domains, Affibodies® and also aptamers, aptamer structures or aptamer sequences and, in particular, photo aptamers (Smith, Collins et al. 2003).

Photo aptamers, in principle, however, any other probe as well which is provided with at least one photo reactive group, offer the advantage, for example, that an analyte which is not bound covalently becomes bonded covalently to the probe as the result of a photo reaction. In the case of heterogeneous test systems, in particular, this makes stringent washing possible such that nonspecific bonds are reduced. This ultimately leads to a higher degree of sensitivity.

In this connection, it is particularly advantageous if the molecular switch is bound to a solid phase, for example to particles, in particular magnetic particles, or to the surface of a reaction vessel such as a microtiter plate. Separation of the solid phase from the liquid phase makes it possible to carry out efficient washing steps.

In addition to Affibodies®, it is also possible to use other antibody-like molecules and their derivatives such as designed repeat proteins (Forrer, Stumpp et al. 2003) and antibody-like protein scaffolds such as anticalins or duocalins (Skerra 2000; Skerra 2001). Designed repeat proteins or protein scaffolds can be prepared, for example, from ankyrins or leucine-rich repeats. Anticalins or duocalins are correspondingly altered lipocalins. It is likewise conceivable to introduce ligands, such as antigens, substrates, cosubstrates, inhibitors, prosthetic groups, etc., into the probe as structural elements possessing affinity. The respective structural elements or components of the probes are correspondingly conjugated to each other.

The binding components or binding partners are involved in the binding of an analyte. By contrast, the coupling components are used to ensure stable bonding of the different components of a molecular switch. The linkage, i.e. chemical bond or conjugation, which is produced by the coupling components can be either covalent or noncovalent. According to the invention, all possibilities of covalent and noncovalent bonds can be employed in this connection.

Suitable covalent bonds include bonds by way of methylenes, methines, ethers, thioethers, carboxylic esters, amides, amines, Schiff's bases or azomethines, enamines, etc. Preference is given to synthesizing linkages which are obtained simply, rapidly and economically by means of the customary coupling reactions (Becker, Berger et al. 1993). In this connection, it is possible to couple reactive groups of the components directly to each other or by way of coupling components or crosslinkers, or, where appropriate, also by way of binding partners such as avidin and biotin. Crosslinkers containing appropriate functional groups such as aldehydes, imidates, in particular imidoesters, carboxylic anhydrides, carbodiimides, succinimide esters, in particular N-hydroxysuccinimide esters, maleimides, haloacetyls, pyridiyl disulfides, hydrazides, isocyanates, glyoxals, etc., or photo activatable functional groups such arylazides, can be used for this purpose. Both homofunctional crosslinkers such as glutardialdehyde and dimethyl suberimidate and heterofunctional crosslinkers which possess different functional groups can be used (Pierce catalog) (Becker, Berger et al. 1993; Rehm 2002). Other chemical modifications of the covalent bond between the catalytic component and the probe component are also conceivable. Bonds by way of Schiff's bases can, for example, be reduced to more stable amines (Becker, Berger et al. 1993).

The noncovalent bonds can be based on Van der Waals forces and on hydrophobic and ionic interactions as well as on hydrogen bonds and coordinate bonds. According to the invention, it is possible, therefore, to use, for example, metal chelate complexes such as nickel-histidine chelates, and others. In addition, it is also possible to use the noncovalent bond of ligands to macromolecules such as that of biotin to avidin or streptavidin and also that of digoxigenin to corresponding antibodies. It is furthermore possible to conceive of providing the probe with an enzyme substrate or enzyme cosubstrate, a cofactor or a prosthetic group and thereby binding the probe to an enzyme which functions as catalytic component.

In addition, it is also possible to conceive of using interactions of what are exclusively low molecular weight substances. In this connection, reference may be made, for example, to the complexing of different substances, preferably salicylhydroxamic acid by 1,2-phenyldiboronic acid (Stolowitz, Li et al. 2002).

In addition, it is also possible to use biotechnologically altered proteins or enzymes for coupling probe and catalytic component. In this case, fusion constructs of appropriate expression systems with, for example, tags such as protein kinase A, thioredoxin, cellulose binding domain, His, Dsb, glutathione-S-transferase, NusA, etc., come into consideration. In addition, it is also possible to use proteins which have been altered by site-directed (molecular modeling) or random mutagenesis. In this connection, proteins which have been altered by mutagenesis have the advantage that functional groups can be introduced at particular sites, thereby making it possible for the components to be linked optimally. In this case, cell-free in-vitro translation systems, in contrast to in-vivo translation systems, should preferably be used, where appropriate, since, in these systems, unnatural amino acids, which permit very specific coupling reactions, can be introduced at particular sites.

The analytical method according to the invention is described in more detail below:

The novel molecular switches can be employed for analytical purposes using appropriate methods. In the methods, at least one molecular switch is brought into contact with a sample to be analyzed. As a result of suitably selecting the experimental conditions, such as temperature and/or the composition of solvent, an analyte (the substance to be detected) which may, where appropriate, be present in the sample, binds specifically to the probe component of the molecular switch. This binding process alters the conformation of the probe and, as a result, the structure and catalytic activity of the catalytic component, and also ultimately of the molecular switch, as well. Since the crucial conformational changes of the probe are based on the hybridization state of the nucleic acid components, it is necessary to select appropriate temperature and solvent parameters in order to permit optimal hybridization processes.

In principle, at least three different scenarios are conceivable. In the first place, while the composition of the medium or the solvent, and the temperature, remain constant, the presence of the analyte can lead to its being bound to the probe. In the second place, while the composition of the medium or the solvent remains constant, at least one temperature change can alter the stability and/or conformation of the probe such that an analyte can be bound to the probe. In the third place, while the temperature remains constant, at least one change in the composition of the medium or the solvent can alter the stability and/or conformation of the probe such that an analyte can be bound to the probe. In principle, it is also possible to combine these scenarios. The result of the scenarios is the binding of the analyte to the probe and thus a change in the conformation of the probe such that a measurable change occurs in the activity of the molecular switch. When temperature changes are used, it is possible, for example, to melt probe regions which are hybridized to each other by means of increasing the temperature continuously or step-wise. Conversely, a reassociation of homologous probe regions can take place as a result of the temperature being lowered continuously or step-wise. In this connection, the presence of an analyte alters the melting process or else, in particular, the reassociation of homologous probe regions, with this ultimately leading to a change in the activity of the molecular switch and to the analyte being detected.

The temperature dependence of the melting and reassociation of homologous probe regions is in turn dependent on the solvent, in particular on the presence of particular salts and organic components as well as their concentrations, and on the composition of the probe, in particular the nucleic acid base composition and the length of the homologous regions. In addition, the concentration of the probes and thus of the molecular switches, and also, where appropriate, of the analytes which are present, have also to be taken into consideration from the point of view of kinetic aspects. Since kinetic aspects also play a role in hybridizations, it is also possible to influence the process by an appropriate choice of incubation times.

Kinetic aspects are also decisive in connection with the hybridization processes especially because the melting of a double-stranded nucleic acid takes place in accordance with a 1st order reaction while the association or reassociation is described by a 2nd order reaction (Smith, Britten et al. 1975; Galau, Britten et al. 1977; Torsvik, Goksoyr et al. 1990; Torsvik, Daae et al. 1998). Consequently, the melting of a double-stranded probe takes place, for example according to

d[single-stranded probe]/dt=k₁[double-stranded probe] (1)

only in dependence on the concentration of the intramolecular double-stranded probe and the 1st order rate constant k₁.

By contrast, an unhybridized probe associates with a single-stranded nucleic acid, i.e. the analyte, to form the probe/analyte complex (probe×analyte) in accordance with

d[probe×analyte]/dt=k₂[single-stranded probe][single-stranded analyte] (2).

Both the concentration of the single-stranded probe and the concentration of the single-stranded analyte have an influence on the time-dependent generation or concentration of probe/analyte complexes.

The rate constants have to be determined empirically and depend on the reaction conditions such as the temperature, the solvent, etc. Since the activity of the molecular switch depends on the concentration of the probe/analyte complex but the time-dependence of the formation of this complex and, where appropriate, the achievement of an equilibrium depend on a large number of parameters such as the temperature, the solvent, the concentration of the probe, the concentration of the analyte, etc., it is not possible to provide any procedural instructions which are generally valid. These depend on the given system. It is also clear, however, that the process, or the respective constituent processes, can be influenced or controlled by way of the incubation time.

Aside from continuously increasing or lowering the process temperature, the temperature in the analytical process is preferably changed in discrete steps. For example, starting from a given initial temperature, the temperature is then increased as rapidly as possible so as to ensure that the appropriate probe regions and, where appropriate, the analytes as well, are present in the “melted”, that is the single-stranded, conformation. In a further step, the temperature is then reduced as rapidly as possible down to a hybridization temperature. Homologous regions then associate or reassociate at this temperature. Where appropriate, the activity of the catalytic component can also be determined at this temperature. It is also conceivable to once again lower or raise the temperature for the activity determination. However, in this connection, the temperature must be below the range of the melting process temperature.

In principle, the process of melting and reassociation can also be controlled by changing the solvent, or else the thermal process steps can be supported by doing this. For example, the melting temperature can be altered, for example lowered, by adding given quantities of at least one organic solvent, an acid or a base and an auxiliary substance. The melting temperature can be raised once again by diluting appropriately with water. A change in the ionic strength, for example by diluting with water or by adding salts, can be used in a similar manner. In this connection, increasing the ionic strength mainly leads to the melting temperature being increased.

Accordingly, different temperatures and/or solvent compositions can be set before, during and after hybridization processes in order to induce appropriate structural changes and/or in order to obtain an appropriate catalytic activity. In this connection, the solvent preferably comprises water and/or organic solvents and can additionally contain, as further components, buffers, salts, substrates, cosubstrates, cofactors, inhibitors of the catalytic component, additional catalysts, in particular enzymes, and auxiliary substances. A particular embodiment also provides for different substrates of differing size and/or affinity to be employed at the same time. This can also relate to the cosubstrates, cofactors and inhibitors. The respective components can all be already present in the solvent at the beginning of the process or be added successively in discrete process steps.

In the case of systems which are initially catalytically active and become inactive as a result of the presence of an analyte, or alter their specificity, it can be advantageous to start the actual reaction after the hybridization processes have come to an end. This can be effected by adding at least one reactant which was still lacking. This reactant can, for example, be a substrate, cosubstrate or cofactor.

Examples of suitable organic solvents are acetone, methanol, ethanol, isopropanol, acetonitrile, etc., and, in particular, dimethyl sulfoxide and formamide. It is possible to use the customary buffers, at different concentrations and also combinations, for the purpose of maintaining the pH stably in a particular range. Examples of these buffers are citrate, phosphate, preferably, however, tris(hydroxy-methyl)aminomethane, solutions.

Aside from the buffering agents, salts, the salt type and the salt concentration, naturally also influence the system. Alkali metal and alkaline earth metal salts such as sodium chloride and magnesium chloride are preferably employed in the hybridization reactions. It is also possible to conceive of using moderate concentrations of chaotropic agents such as guanidinium salts (guanidinium chloride, guanidinium hydrochloride, guanidinium thiocyanate, guanidinium isothiocyanate, guanidinium dodecylsulfate, etc.) or urea. Where appropriate, the salts are also necessary for enabling catalysis to take place. Magnesium salts and other bivalent cations are sometimes required for the enzymatic transformation of substrates. The latter, and also their cosubstrates and cofactors, can naturally also be present in the solvent.

Aside from the substrates, cosubstrates and cofactors of the actual catalytic component of the molecular switch, such substances can also be present in the case of additional catalysts. Additional catalysts, in particular enzymes, can be employed when carrying out coupled tests, for example by means of redox cycling (Bergmeyer 165; Bisswanger 1994).

Auxiliary substances, such as redox-active substances, for example dithiothreitol, glutathione, thioctic acid and β-mercaptoethanol, can also be employed for the purpose of stabilizing the components or reaction sequences. The addition of proteins such as albumins, for example bovine serum albumin, can likewise have an advantageous influence on individual process steps. Ectoins (Rehm 2002), which stabilize the structures of proteins against heat, in particular, can also be used in order to prevent or reduce the denaturation of proteins at higher process temperatures.

However, auxiliary substances can also be compounds which interact in a particular manner with the nucleic acids of the probe component of the switch and thereby have an influence on the structure and the hybridization behavior of the probe or of the analyte. Examples of these compounds are cyanine dyes, phenanthridines, acridines, indoles, imidazoles, actinomycins, hydroxystilbamidines (Haugland 2002) and also ornithines and spermidines.

The above-listed influences on the conformation of the probe and the binding of the analyte are complex. For this reason, the optimum conditions have to be determined for each individual case. A more detailed description of the above-listed influences, and of the methodological adaptations, in particular with regard them, is given below.

In the case of hybridization events involving nucleic acids and their derivatives, the process is usually a reversible process which is influenced by a variety of factors. These factors include the percentage content of the guanine and cytosine bases (% GC), the length of the nucleic acid(s), the concentration of monovalent cations such as sodium and agents, such as formamide, DMSO, etc., which have an influence on the stability of the nucleic acid single strand or the nucleic acid double strand. These factors are connected to each other in accordance with a formula which was determined empirically for DNA (Meinkoth and Wahl 1984). This formula can be used to calculate the melting temperature (Tₘ) of a double strand:

Tₘ=81.5° C.+0.41(%GC)+16.6 log [Na⁺]−500/n−0.61(% formamide) (3).

Following (1), account can also be taken of the formula originating from Howley et al. (Howley, Israel et al. 1979). In this case, the mismatch proportion is also taken into account:

Tₘ=81.5° C.+0.41(%GC)+16.6log [M+]−500/n−0.61 (%F)−1.2D (4),

where % GC=percentage content of G/C pairs, [M⁺]=concentration of monovalent cations, n=number of nucleotides, % F=percentage content of formamide in the buffer
D=percentage mismatch content.

However, in practical use during recent years, it has been found that the melting temperature which is calculated using these formulae is not to be regarded as absolute but, instead, only provides a suitable point of reference. Presumably, DNA double strands do not behave in situ as they do in solution (Leitch and Heslop-Harrison 1994). According to Britten and Kohne (Britten and Kohne 1968), the optimum hybridization temperature or process temperature can be calculated from the melting temperature which is computed using (3) as follows:

Tₕ=Tₘ−25° C. (5).

It is also possible to have recourse to the Wallace rule, particularly in the case of relatively short oligonucleotides such as primers:

Tₘ=2° C.×(A+T)+4° C.×(C+G) (6)

It is clear from this equation that the Tₘ value depends on the length and sequence of the oligonucleotide. However, this rule was drawn up, in particular, for hybridizations to membrane-bound oligonucleotides and is based on a salt concentration of 1 M. For solution experiments, 8° C. should be added to the computed temperature.

The nearest neighbor method is also available. It also takes into account the sequence-dependent stacking effects when calculating the Tₘ values and is based on the thermodynamic data of adjacent nucleotide pairs. This method gives reliable values for medium-length oligonucleotides (20-60 bases):

Tₘ=[(1000×dH)/(A+dS+R×In(C/4))]−273.15+16.6×log c(K+) (5),

where dH=sum of the enthalpies of the pairs, dS=sum of the entropies of the pairs, A=−10.8 cal, entropy of the helix formation, R=1.984 cal/degree×mol, gas constant, C=oligonucleotide concentration (250 pmol/l), c(K+)=concentration of the potassium ions in the oligo solution (50 mmol/l).

In summary, the influence of the temperature and of the solvent can, for example, be described by

Tₘ=81.5+0.41(%

GC)+16.6 log c(M+)+x log c(M⁺⁺)+n log c(Mⁿ)−500/n−

0.61(%F)−d(% auxiliary substance)−1.2D (2),

following on from (1) and (2). In this case, the influences of additional ions (M⁺⁺; Mⁿ) having different valencies are also taken into account. Auxiliary substances as described above, that is additional organic solvents and other additives, are also taken into account. Where appropriate, the influences of different ions, for example by way of the ionic strength, can also be combined in a term. Formamide and other additives can be dealt with in a corresponding manner.

The use of the analytic system according to the invention to detect the analyte is described in more detail below:

A variety of detection methods can be used to measure the extent of the catalytic activity and specificity (or its change) of the molecular switches such that the presence (qualitative) and the concentration (quantitative) of an analyte can be determined. The analytical test system which is based on the molecular switch can be employed in a flexible manner. Basically, the molecular switch enables a binding event to be recorded. In this connection, the nature of the recording depends on the type of reaction which takes place.

Since energetic changes occur in connection with almost all described processes, for example in connection with enzyme reactions, it is in principle possible to employ calorimetry or microcalorimetry using calorimeters containing reaction vessels which are envisaged for this purpose. Optical measurements can be carried out if there is a change in the spectral properties of the solution, for example as a result of the involvement of luminescent, in particular fluorescent, and absorbent compounds, particularly in connection with enzyme-catalyzed reactions. In this case, optical measurements comprise luminometry, fluorimetry, photometry, polarimetry, polarometry, etc. using the appropriate equipment and reaction vessels. In principle, it is also possible to detect visually, i.e. when, for example, using test strips, simple cuvettes or microtitration plates, etc. It is likewise possible to conceive of radiometric methods when radionucleotides are used in reactions. Methods, such as manometry, which record pressure differences can also be employed. This is of interest when osmotic processes take place and also when gases are formed or consumed, for example when using decarboxylases. Amperometric methods and corresponding apparatus, as are used, for example, in polarography, are to be employed in connection with electrochemical processes using electrodes. This also includes determining potential differences, currents, impedances, etc., and changes in these parameters.

Appropriate reaction vessels, such as cuvette systems, microtiter plates and filter strips, and also arrays, chips, beads, etc., are to be employed in dependence on the reaction which is carried out and on the detection method and analyzer which are associated therewith.

Both absorption measurements and luminometric measurements can be used in the spectrophotometric methods. Absorption measurements comprise, for example, detecting chromophores and determining turbidities. The latter can also be determined using scattered light or reflected light measurements. Fluorescence, phosphorescence, chemoluminescence and bioluminescence measurements are, for example, available in connection with luminometric measurements.

According to the invention, electrical measuring methods can be used in connection with certain redox processes. Determinations of the current flow, of the voltage or of the resistance, where appropriate of the frequency-dependent resistance (impedance) as well, are all equally suitable.

In general, both kinetic measurements and end point determinations are conceivable. Both individual wave-lengths and spectra can be recorded in the case of spectrophotometric measurements, in each case depending on the system employed.

Detection systems which use coupled tests may be of particular interest. The reaction of the catalytic component can be coupled to another reaction in order to facilitate detection and, where appropriate, increase sensitivity. Redox cycling may be of particular interest in this connection. For example, the pyridine dinucleotide-dependent reaction of a catalytic component can be supported by using a selected enzyme, a dehydrogenase (formate dehydrogenase, diaphorase, etc.), to regenerate the corresponding pyridine nucleotide. Where appropriate, the enzyme which is additionally introduced can also catalyze the actual detection reaction. It is likewise conceivable to integrate further enzymes into this process.

If electrodes are integrated in the system as catalytic component, or part of a catalytic component, of the molecular switch, or as additional components, it is also possible to take advantage of electrically supported redox cycling, which can also, where appropriate, be used for electrical detection. For example, p-aminophenolgalactose (as analyte) can be cleaved by β-galactosidase (as catalytic component) and the resulting p-aminophenol can be oxidized to quinoneimine by way of an anode. The reduction of the quinoneimine at a cathode in turn yields p-aminophenol, such that a sensitive electrical detection can take place over several cycles. In addition, the electrochemical detection can be effected, for example, by way of the hydrogen peroxide-dependent conversion of hydroquinone into benzoquinone using a peroxidase (horseradish peroxidase). While consuming protons, hydroquinone is regenerated from benzoquinone at a cathode.

The bases for the change in the catalytic activity are described below:

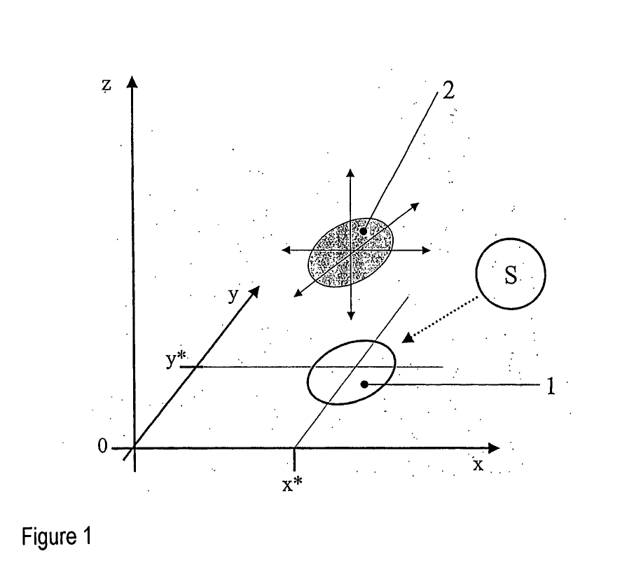

A possible basis for the change in catalytic activity is depicted in FIG. 1. The catalytic component, or access to the catalytic component (1), is crucial for transforming a substrate (S). The access of the substrate to the catalyst is determined both by the geometry and size of the substrate itself and by the geometry and size of the catalytic component and of the probe. The position of the catalytic component in relation to particular regions of the probe (2) is also crucial.

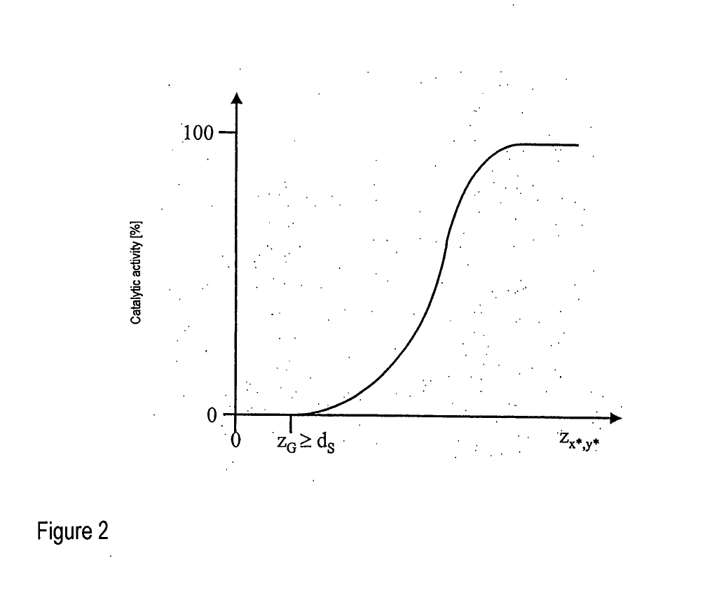

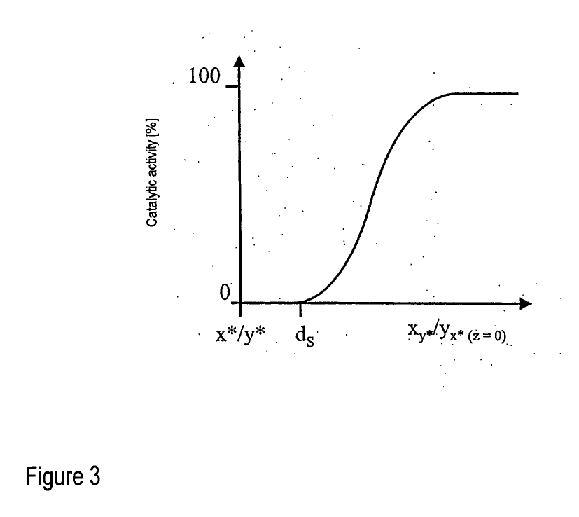

Assuming the relative dimensions and geometries specified in FIG. 1, and assuming that there are no Van der Waals and electrostatic interactions between the individual components, it is then possible to derive the local slope functions of the catalytic activity which are depicted in FIGS. 2 and 3. In this connection, both the probe and the access to the catalytic component are defined, for simplicity, as circular areas having equal radii. The substrate is ascribed a spherical geometry. If the probe is located in the x* and y* position, the earliest point at which the substrate can gain access to the catalytic component is at a limit distance (z_{G}) which has to correspond to at least the diameter of the spherical substrate (dₛ). Since a significant diffusion limitation (Bisswanger 1994) by the probe is still to be expected at short distances between the probe and the access to the catalytic component, the catalytic activity only increases gradually at first in order subsequently approach a maximum (FIG. 2).

This naturally only applies when the probe is in the x* and y* position. The dependency of the system on the space coordinates x or y becomes clear when z is made equal to zero in the limit case and the probe comes to lie directly over the access in a superimposable manner. Displacing the probe in the y direction while the x* position remains constant, or vice versa, gives, in accordance with the conditions and assumptions from FIG. 2, the slope function which is depicted in FIG. 3.

Ultimately, the catalytic activity results from the probability with which a substrate has access to the catalyst or catalytic centre and thus from the super position of the influences of all the space coordinates, the catalytic components, the probe and the substrate. Even if electrostatic and Van der Waals' interactions have been disregarded in this analysis, they still have an effect. They should not, however, have any significant effect on the course of the slope functions.

Since, now, drastic differences in activity can be achieved by varying the spatial conditions in relatively narrow ranges, it is outstandingly suitable to use the molecular switch according to the invention for detecting an analyte. The binding of an analyte to the probe alters the position of the relevant regions of the probe in relation to the catalytic component, and thus also the activity of the entire system, to such a significant degree as to make it possible to detect analytes specifically and sensitively.

In the example embodiments which are described below, the molecular switch (3) usually comprises a probe (4) which is conjugated to the catalytic component (5) either directly or by means of a coupling component (6). The access to the catalyst (1) is limited by the structure or surface (9) of the catalytic component (5). This structure can be a support, a membrane or a matrix with which the molecular switch can be associated or to which it can be fixed (immobilized molecular switch).

Carriers can, for example, be glasses, topazes, polymers, plastics, ceramics and other composite materials which are provided with pores which permit access to the actual catalyst. It is likewise possible to conceive of this support being coated, for example with membranes or matrices, in order to generate appropriate pore structures and/or in order, where appropriate, to permit coupling of a probe component. Reference may be made, in this connection, to photolithographic processes or etching techniques.

It is also conceivable that, instead of an access to a catalyst, the catalyst itself is brought into this position. This is conceivable, for example, in connection with using metal catalysts or electrodes as catalytic component. Surfaces at which the carrier exhibits discrete metallic regions come into consideration, for example, in this connection.

The structure or surface (9) which is depicted can naturally also, for example, be the surface of an enzyme which surrounds the access to the catalytic centre or a binding site for an allosteric ligand. In the general manner, this structure (9) can correspond to any matrix which separates the probe region from the catalyst region in the switch or surrounds the access to the catalyst or to the catalytically active centre. In this case, the molecular switch is as a rule not immobilized or is located in the mobile phase of the reaction mixture.

The analyte is preferably located in the liquid or mobile phase of the reaction mixture or test mixture.

### EXEMPLARY EMBODIMENT FOR UNHYBRIDIZED SINGLE-STRANDED PROBES

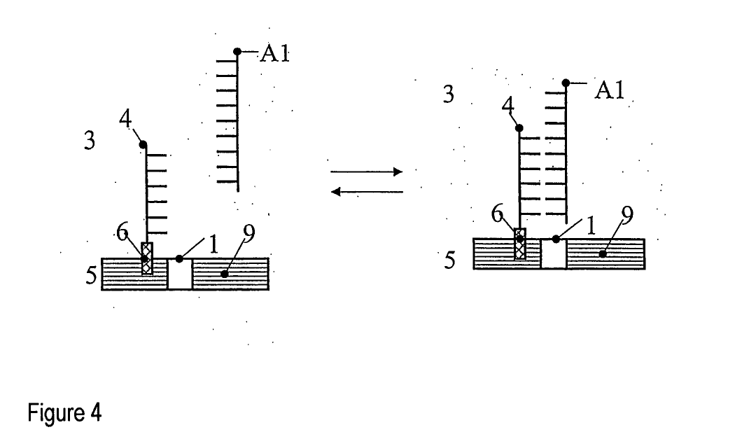

In the exemplary embodiment using a single-stranded probe which is not initially hybridized (FIG. 4), the molecular switch (3) is composed of a single-stranded probe (4) which can be associated with the catalytic component (5) directly or, where appropriate, by means of a coupling component (6). Under appropriate hybridization conditions, the structure of the molecular switch having a single-stranded probe enables an analyte (A1), for example a single-stranded RNA or DNA, to be bound to the probe component (4). If an analyte (A1) hybridizes, the access to the catalytic centre (1) becomes restricted. As a result, the catalytic activity of the system falls, with this being used for detecting the analyte. If this system is to be used for detecting double-stranded nucleic acids, such as double-stranded DNA, the latter must first of all be denatured. The homologous strand then hybridizes with the probe component (nucleic acid).

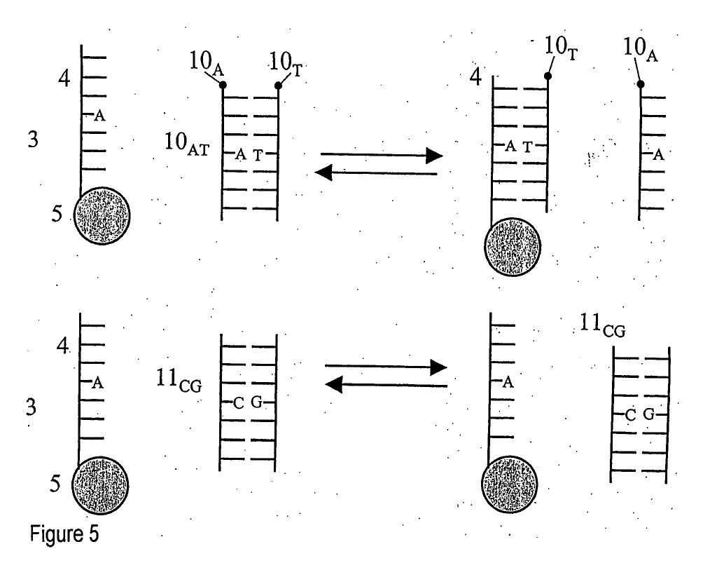

The system having a single-stranded probe which is initially unhybridized is also suitable for detecting point mutations (single nucleotide polymorphisms (SNPs)) (FIG. 5). Under appropriate hybridization conditions, only the homologous strand (10T) of the analyte (10AT) hybridizes the corresponding homologous probe (4) of the molecular switch (3). By contrast, it is not possible for a strand of the analyte (11CG) to bind, which means that the AT/CG SNP in the cited example can be detected. A, T, C and G are chosen in analogy with the customary base designations in nucleic acids.

### EXEMPLARY EMBODIMENTS FOR PROBES WHICH ARE INITIALLY PRESENT IN HYBRIDIZED FORM

Probes having intermolecular secondary structures are described first of all:

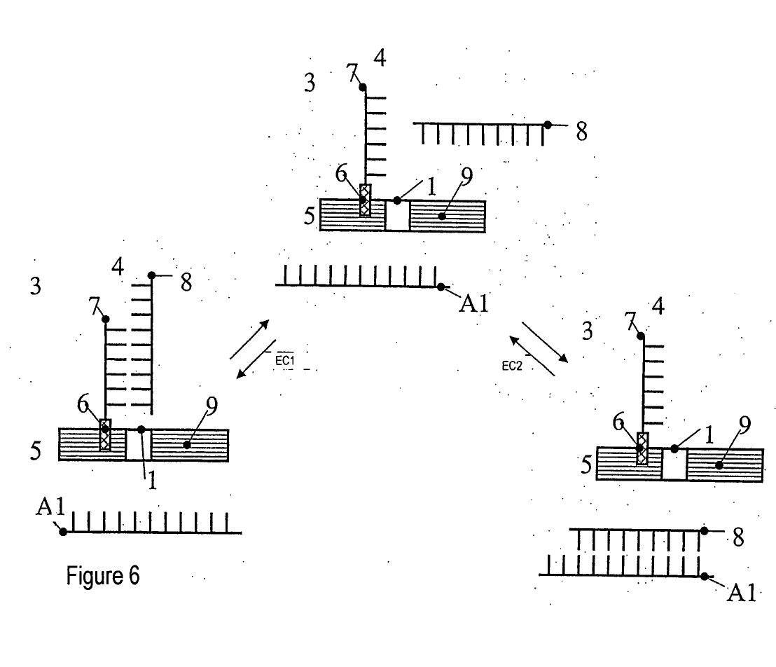

In systems having a hybridized, double-stranded probe (FIG. 6), the molecular switch (3) comprises a probe (4) which can be linked to the catalytic component (5) either directly or, where appropriate, by means of a coupling component (6). In this connection, only a particular (part) component (7) of the probe (4) is conjugated with the catalytic component (5). A bound homologous (part) component (8) of the probe (4) restricts the access (1) to the catalyst.

In this connection, the probe (4) is preferably to be selected such that the binding of the probe component (8) to the analyte (A1) is more stable than the binding of the probe components (7) and (8) to each other. This can be achieved by the hybrid composed of the probe component (8) and the analyte (A1) comprising more homologous base pairs than the hybrid of the two probe components (7) and (8). As a result, the stability or melting temperature of the hybrid between the probe component (8) and the analyte (A1) is higher than that of the probe (4). Firstly, the probe components (7) and (8) are separated from each other by appropriately raising the temperature and/or in some other way altering the experimental conditions (VB1), for example the salt concentration. A further change in the experimental conditions (VB2), for example a lowering of the temperature, then leads to the association of the probe component (8) with the analyte (A1) before reassociation of the probe components (7) and (8) can take place since the association stability or the melting temperature or reassociation temperature of the hybrid of the probe component (8) with the analyte (A1) is higher than that of the probe (4), i.e. of the probe components (7) and (8) with each other. The specificity and sensitivity of such a system is greater than of a system in which the melting temperatures are comparable.

If double-stranded nucleic acids, such as double-stranded DNA, are being detected, the aspect of the stability of the hybrid of the probe component (8) and the analyte (A1) being higher than the stability of the probe (4) is sometimes decisive.

As a result, the association, with the probe component (7), of the strand which is homologous with the analyte is reduced or prevented. Such an association would mainly lead to an exchange of the hybridization partners and thus not permit any effective detection of the analyte since, in this way, there would be no change in the conformation of the probe and thus no change in the catalytic activity of the catalytic component.

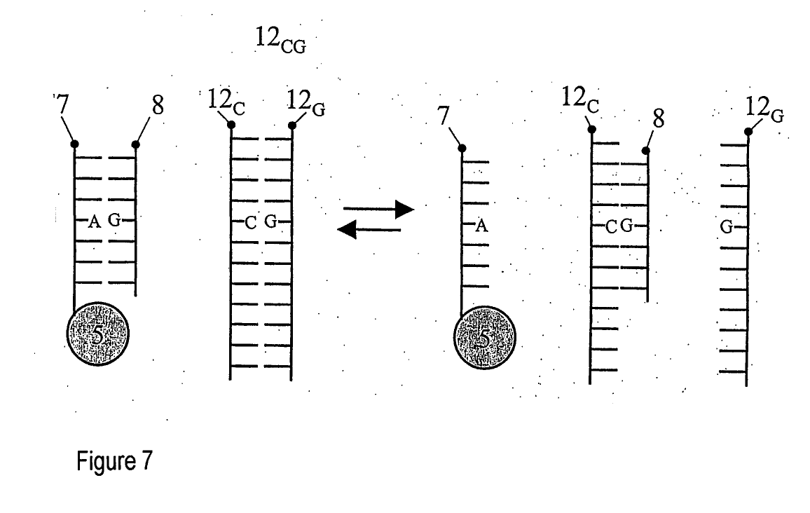

Instead of using probe components (7) and (8) which are of differing lengths, it is likewise possible to conceive of using those which are of the same length but which exhibit at least one mismatch (FIG. 7). This means that no base pairing takes place at least one site within the homologous regions. This mismatch should preferably be located in the central region of the probe (4). This mismatch consequently destabilizes the probe (4) with regard to the corresponding region of the analytes (12). In this connection, the probe mismatch is to be selected such that while, under appropriate experimental conditions, the probe component (8) hybridizes with the homologous 12_{C} strands which are to be detected, the 12_{G} strand does not hybridize with the probe component (7). This can be ensured, for example, by selecting suitable rehybridization temperatures. While it is true that the probe component (8) and the strand 12_{G} will compete for binding to the analyte stands 12_{C} during the rehybridization process, this composition process provides corresponding ratios of molecular switches of differing activity, which ratios can then be determined. Furthermore, the rate at which short probe components 8 reassociate with the 12_{C} analyte strands is higher than the rate at which the 12_{G} analyte strands reassociate with the 12_{C} analyte strands. As a result, it is chiefly hybrids composed of probe component (8) and strand 12_{C} which will be formed.

### Probes Having an Intramolecular Secondary Structure:

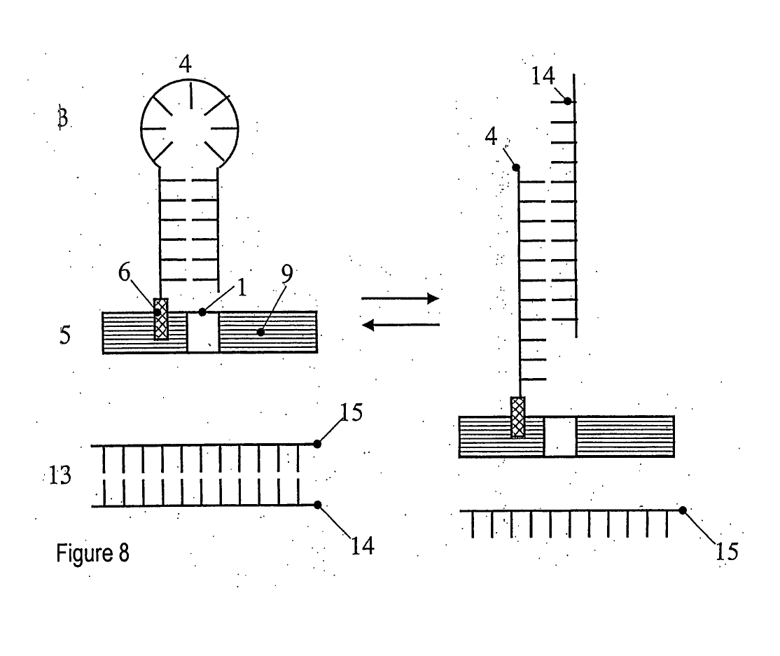

In addition, using the probe structure which is depicted in FIG. 8 can be of help for avoiding hybridization processes which interfere. In this case, the probe (4) is merely composed of one probe component which is bound to the catalytic component (5) and which possesses a region which is hybridized intramolecularly. In the presence of an analyte (13), the appropriate homologous structure (14) will hybridize with the probe, thereby dissociating the intramolecular hybridization of the probe. This thereby enables any possibility of strand (15) of the analyte hybridizing with the probe (4), and thereby interfering, to be ruled out.

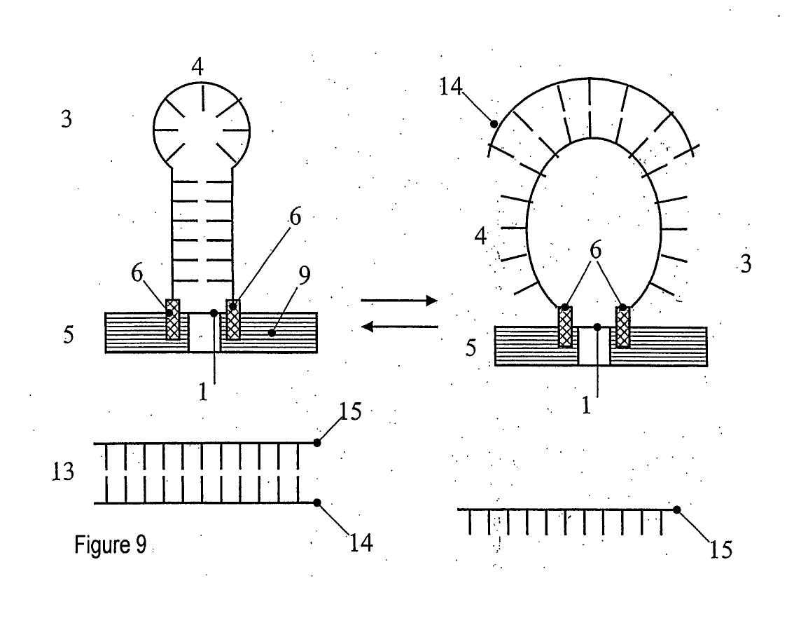

In one particular embodiment (FIG. 9), the probe (4) having an intramolecular secondary structure (e.g. a nucleic acid) is conjugated, either directly or, where appropriate, by way of coupling components (6), with the catalytic component (5) both at the 5′ end and at the 3′ end. In its intramolecularly hybridized, closed state, the probe restricts access to the catalyst (1). By contrast, the open state of the probe, in which the probe is hybridized intermolecularly with the analyte, offers a substrate better access (1) to the catalyst (5).

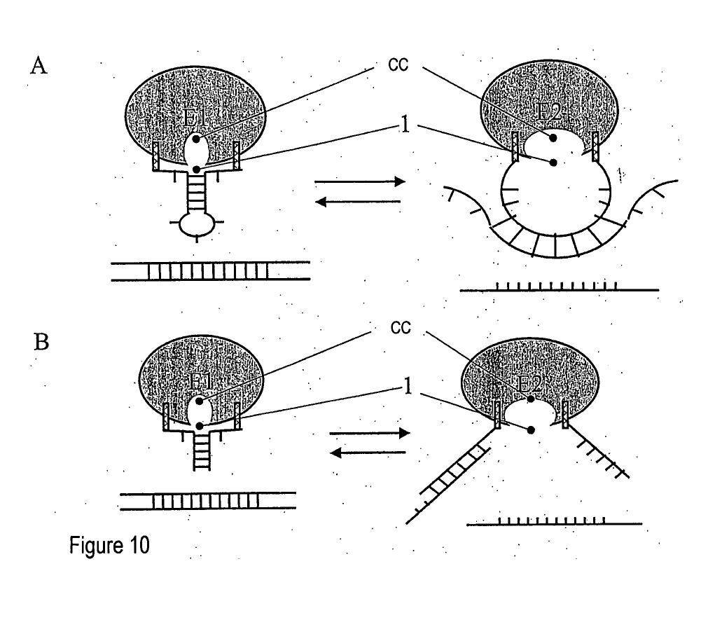

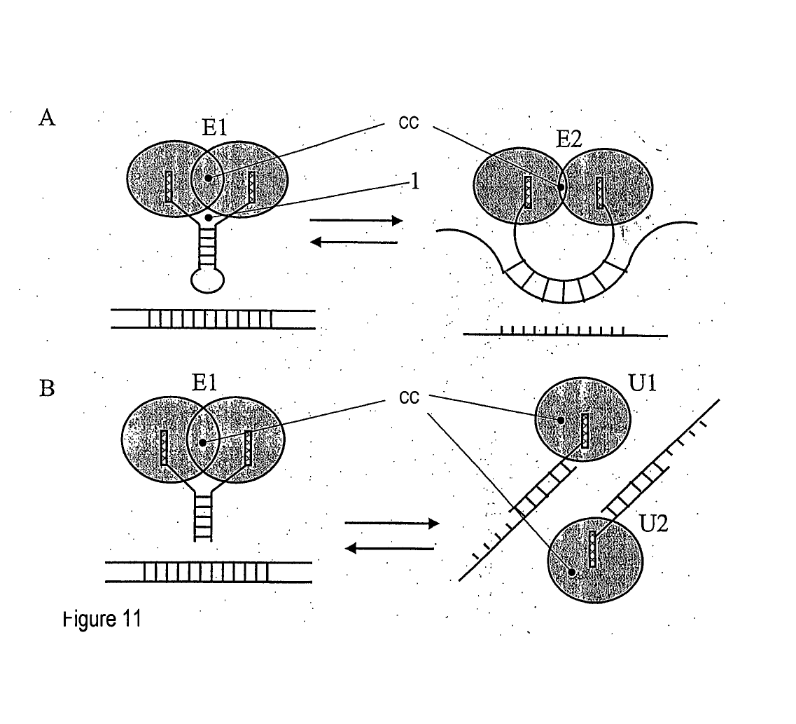

If the catalytic components (5) are enzymes (Es) (FIGS. 10 and 11), other effects aside from the influence on the access to the catalytic centre (1), such as conformational changes (CCs) at the catalytic component itself, can occur, with these other effects then in turn leading to a change in the catalytic activity. The enzyme (5) is consequently present in at least two forms, E1 and E2, which are of differing catalytic activities. The probe embodiments which are chosen in FIGS. 10 and 11 can naturally also be replaced with other embodiments according to the invention.

These conformational changes (CCs) at the catalytic component of the molecular switch can arise, in particular, in the case of enzyme complexes which are constructed from several subunits. By way of example, FIG. 11 depicts the influence on a diaphorase homodimer whose catalytic activity is determined by the interaction of the subunits (U1 and U2), such that at least two forms, E1 and E2, having different catalytic activities can be present in this case as well.

In one particular embodiment (FIG. 11B), the subunits (U1 and U2) of the enzyme are completely separated in this connection, with this naturally having a particularly dramatic effect on the enzymatic activity and consequently being able to be of particular interest for determining an analyte. In this case, too, the probe embodiment which is depicted can be replaced with other embodiments according to the invention.

Possible additional constituents of the probe component are described below by way of example.

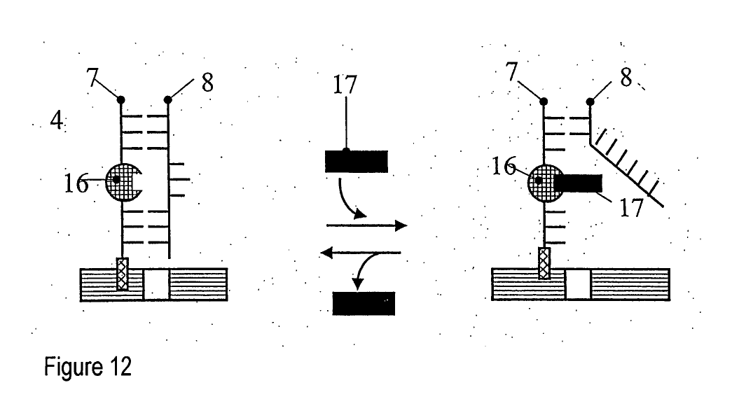

a) Structural Elements Exhibiting Affinity (cf. FIG. 12):

If structural elements 16 exhibiting affinity, such antibodies, Affibodies®, aptamers, etc., are integrated in the probe component (4) (e.g. a nucleic acid) or in at least one probe strand (7 and/or 8) or a region of a probe having an intramolecular secondary structure, it is also possible to detect other analytes (17) in addition to nucleic acids (FIG. 12). The binding of an analyte to the structural element exhibiting affinity partially or completely prevents the probe component 4 from (re)associating, which means that there is free access to the catalyst.

If nonhybridized single-stranded probes are used, homologous strands have to be added to the reaction mixture. These homologous strands then compete with the analytes for binding to the single-stranded probes. If the analyte binds, the molecular switch is catalytically active. If the homologous strand binds or hybridizes, the access to the catalytic centre (1) is then blocked such that the molecular switch will not be catalytically active or will at least be less catalytically active. This thereby results, in dependence on the concentration of the molecular switches, of the homologous strands and, in particular, the analyte, in the system having an overall activity which depends on the analyte concentration.

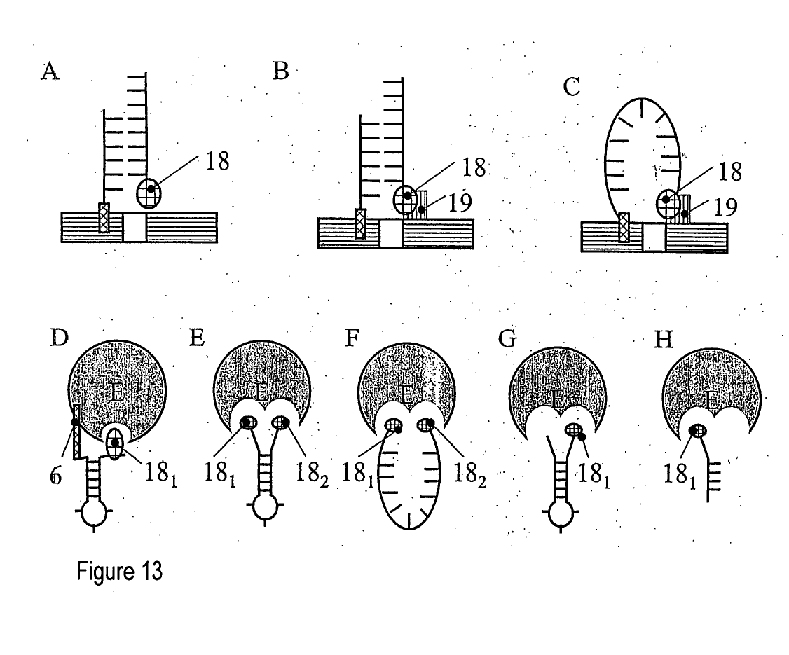

b) Structural Elements Having an Influence on the Access to the Catalytic Component (cf. FIG. 13):

In order to increase the influence on the access of a substrate to the catalytic component, probe components can be conjugated, in appropriate regions, with other components, i.e. blocking components (18) (FIG. 13A). For this purpose, macromolecules such as proteins (albumin, streptavidin, etc.) can, for example, be coupled to the probe at the appropriate sites. It is naturally also possible to conceive of using low molecular weight substances for this purpose.

In particular, the component (18) can in turn be a binding partner for another molecule (19) (FIG. 13B, C). For example, the binding partners can, in this case, be binding partners or complexes comprising ligand/receptor, antigen/antibody, Affibody®, aptamer or substrate, inhibitor/enzyme. If one of the binding partners (19) exhibiting affinity is now linked to the catalytic component (5), this can improve the efficiency of the molecular switch since the access to the catalytic centre is blocked more efficiently as a result. When an analyte binds to the probe, the binding between the binding partners (18) and (19) is severed such that there is once again free access to the catalyst. In the cases mentioned under b), the probe (4) is, in addition, for example, to a coupling by way of a coupling component (6), also preferably connected to the catalytic component (5) by way of the components (18) or (18) and (19).

Due to the characteristic of the binding partners (18) and (19) dissociating when an analyte binds to the probe under the appropriate experimental conditions, these noncovalent binding complexes differ from the conjugation or coupling components (6) which, under standard conditions, bring about a permanent binding of the probe component (4) to the catalytic component (5).

In the embodiment which is depicted, by way of example, in FIG. 13C, it can be seen that an intramolecular hybridization of the probe is not absolutely necessary. It is accordingly also possible to use a nonhybridized, single-stranded probe which, in addition to the direct coupling or indirect coupling through a coupling component (6) is also linked to the catalytic component by way of the binding of the binding partners (18) and (19).

Using such systems would also make it possible to construct competition tests. In this connection, the analyte to be detected is at the same time one of the binding partners (18) or (19). A certain proportion of bindings between the probe components and the catalytic components is thereby prevented in the presence of the analyte, with this having an effect on the catalytic activity of the system such that the analyte can be quantified.

In principle, it is also possible to conceive of replacing the coupling component (6) with a binding of the binding pair (18) and (19) which exhibits a higher degree of binding reversibility. In this case, when completely bound by the analyte, the probe would ultimately be completely separated from the catalytic component.

Using enzymes (Es) gives rise to other possibilities, some of which are depicted in FIG. 13 D-H. Active or allosteric centers of the enzymes, and the corresponding ligands (18) such as substrates, cosubstrates, prosthetic groups, inhibitors, etc., are suitable binding partners in this case. In this connection, the probe is linked to the enzyme by way of at least one ligand (18₁) (FIG. 13 D-H). In principle, it is possible to use any probe structure according to the invention in this connection, i.e. intramolecularly hybridized probes (FIG. 13 D, E, G), intramolecularly hybridized probes and unhybridized probes (FIG. 13 F, H). The probe can be linked to the enzyme, as catalytic component, by way of further coupling components (6) (FIG. 13 D) or further ligands (18₂). Under the appropriate experimental conditions, and due to conformational changes and/or the change in the accessibility to the active centre, the presence of an analyte then leads to a change in the catalytic activity of the enzyme.

According to another embodiment, probe components which are not covalently conjugated to the catalytic component are employed in the following manner. Instead of having the catalytic component, in particular an enzyme, in the assay right from the beginning, the enzyme is only added after the hybridization reaction in which the analyte is bound to the probe. The binding to the catalytic component, and consequently also the activity of the catalytic component, which can be determined by way of appropriate reactions, are influenced in dependence on the hybridization of the probe component with the analyte, in order to detect the analyte qualitatively or quantitatively.

Preference is given, in this connection, to using probe components which, reversibly or irreversibly, possess at least one ligand, such as a substrate, a cosubstrate, a prosthetic group, a allosteric activator or inhibitor and/or a general inhibitor (see FIG. 13 E-H).

For example, an activator which is coupled to a probe component and is hybridized with an analyte is no longer able to activate a catalytic component, in particular an enzyme, as does a free activator probe component. The activity of the catalytic component, which is measured over at least one appropriate reaction, is then used for detecting the analyte. The same applies, in a corresponding manner, to inhibitors. If substrates, cosubstrates and/or prosthetic groups which are coupled to a probe component are used, the respective free and analyte-bound conjugates likewise have different effects on the catalytic activity of the catalytic component and can in this way be used for detecting the analyte.

Membrane systems and matrix systems which can be used as test systems according to the invention are described below by way of example:

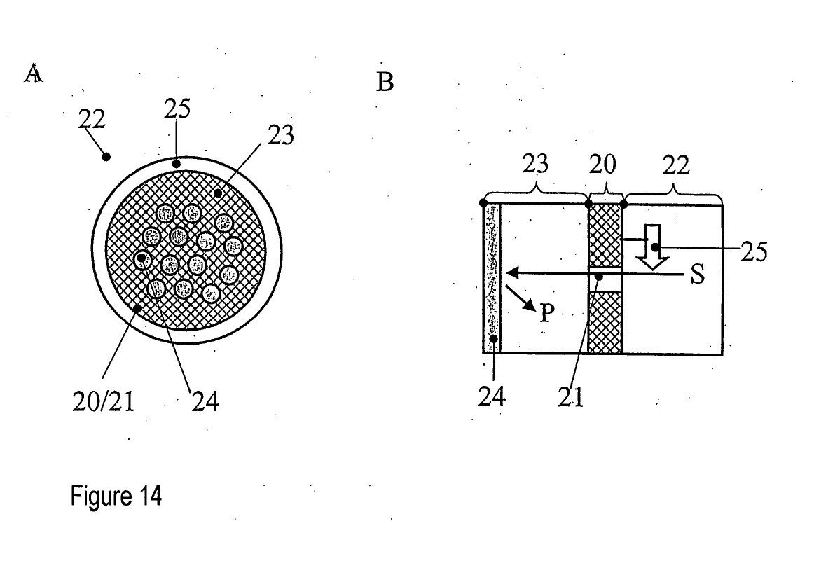

FIG. 14:

In these systems, membranes or matrices (20) which possess appropriate pore structures and permeability properties (21) separate the sample space (22) from the catalyst space (23) in which the catalyst (24) is located. In dependence on the analyte concentration, probe components (25/arrows) in the sample space (22) and/or in the catalyst space (23) influence the transfer of substrates and/or products through the membrane or matrix (20). The transfer, which is consequently dependent on the analyte concentration, of substrate from the sample space to the catalyst space thereby determines the conversion of substrate in the catalyst space. It is naturally also possible to conceive of influencing the transfer of product (reactive substrate) from the catalyst space to the sample space in dependence on the analyte concentration. The respective rates of transfer of substrate and product depend on the properties of the latter, in particular their molecular sizes and charge properties and also hydrophobicity.

FIG. 14 A:

For example, such a system can be composed of catalysts, preferably in the form of enzymes (24), which are immobilized or embedded in a particulate matrix, for example acrylamide or agarose. In this case, the interior of the matrix corresponds to the catalyst space (23) and the surface of this matrix corresponds to a membrane (20) which has a porous structure (21). It is likewise possible to conceive of enclosing catalysts or enzymes in particles without the catalysts or enzymes necessarily having to be embedded in a matrix in the particle interior. The surface of the particles is conjugated with probe components (25) which, in dependence on the analyte concentration, control the transfer of substrate and/or product between the intraparticulate catalyst space (23) and the extraparticulate sample space (22). Suspensions of these particles can be employed for detecting analytes (in the liquid phase of the suspension) in appropriate reaction vessels.

FIG. 14 B:

In contrast to a microscopic separation of sample and catalyst space, it is also possible for these compartments to be separated macroscopically using appropriate reaction vessels. In this case, sample space (22) and catalyst space (23) are separated by a porous membrane or matrix (20) which is provided with probe components (25/arrow). In the depicted example, the probe components (25) control the passage of substrate (S) from the sample space (22) into the catalyst space (23) in dependence on the concentration of an analyte. Depending on the properties of substrate (S) and product (P) (converted substrate), it is naturally also possible to conceive of controlling the transfer of product through the membrane. It is likewise possible to conceive of influencing the transfer of both substances, i.e. substrate and product.

The substrate is converted in the catalyst space (23). In the specified example, the catalyst is integrated or immobilized in the reaction vessel. As long as the catalyst, in particular an enzyme, is unable to pass from the catalyst space (23) into the sample space (22), it is conceivable to use dissolved or suspended catalysts. In this connection, the catalysts can also be dissolved or suspended catalysts or enzymes, etc., which are present in immobilized form.

Depending on its properties, on the membrane and on the probe, the product (P) which is generated either becomes concentrated in the catalyst space (23) (FIG. 14 B) or else also diffuses into the sample space (22). Depending on the system which is used, it can now be advantageous, in this case, to carry out an optical detection, for example in the catalyst space (23) alone. The membrane (20) can prevent the transfer of sample substances into the catalyst space (23) such that interfering “matrix effects” (Zipper, Buta et al. 2003) are reduced. In addition to optical detection by means of absorption or luminescence measurements, the set up which is described naturally also offers the possibility of carrying out comparative osmotic measurements between the sample space (22) and the catalyst space (23) as well as of appropriately using electrical parameters such as current, voltage, resistance and impedance.

In general, the application of a potential difference between the sample space (22) and the catalyst space (23) can be advantageous in this exemplary embodiment since it is in this way possible to make use of electrophoretic aspects. For example, the electrophoretically accelerated transfer of substrate or product through the membrane can have a positive influence on detection.

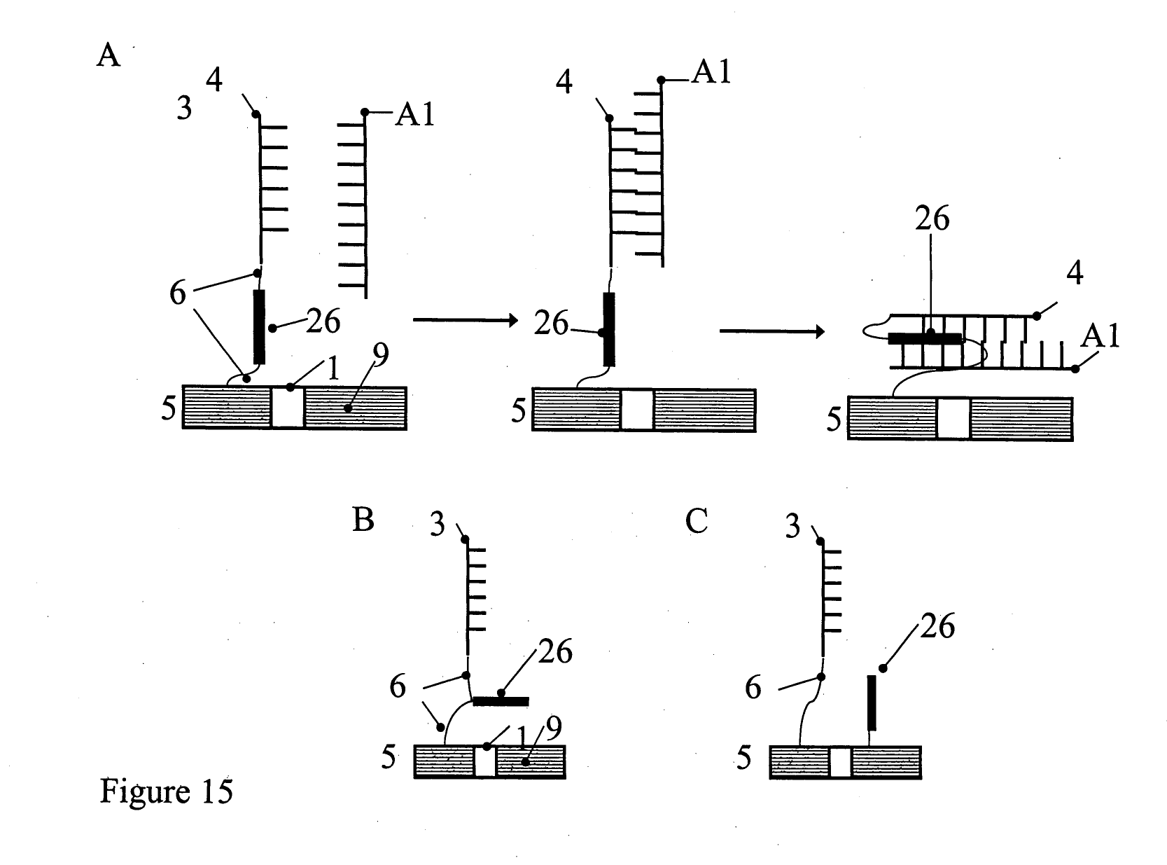

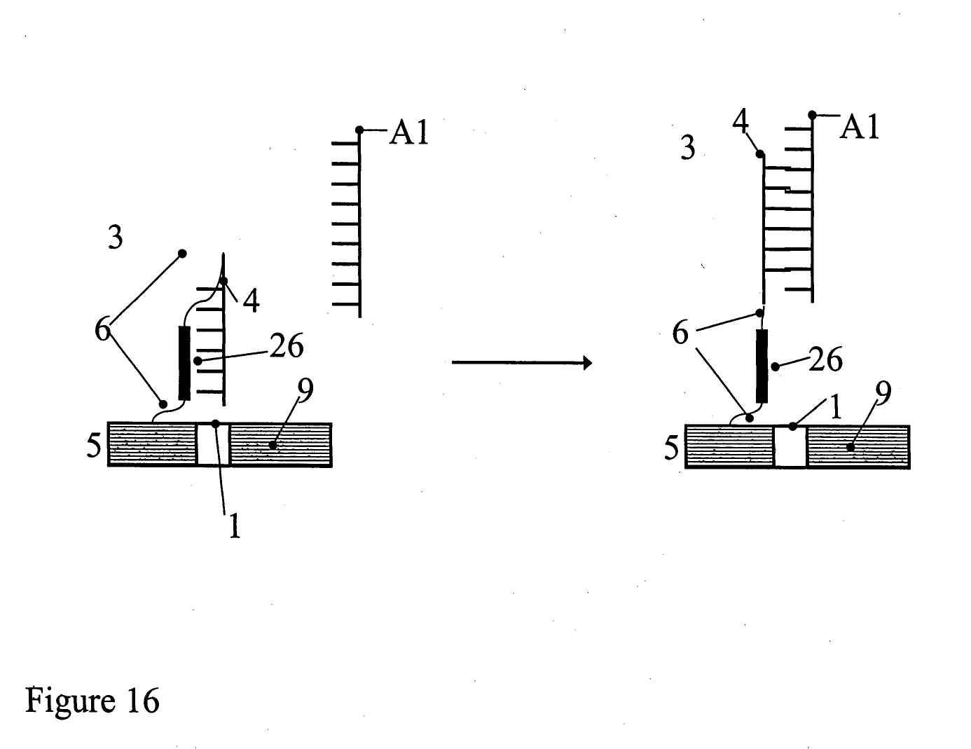

FIGS. 15 and 16 depict a particular embodiment of the analytical test system according to the invention. This test system, which comprises a molecular switch possessing a probe and a catalytic component, additionally integrates at least one component which exhibits different affinities for the hybridized and unhybridized forms of the probe. This so-called selective component (26) can be part of the coupling component (6) (FIG. 15 A, C and FIG. 16) which connects the probe to the catalytic component (5) or with the coupling component (6), the probe (3) and/or the catalytic component (5) (FIG. 15 C).

The selective components can be any chemical compound which is able to bind selectively to hybridized or unhybridized probes, i.e. which exhibits different binding constants or affinities.

For example, the selective compounds can be nucleic acid-binding proteins or peptides which preferably bind to single-stranded or double-stranded nucleic acids or to nucleic acids which are present in triplex, quadruplex or other hybridization states. Examples of proteins which preferably bind to double-stranded nucleic acids are DNA-binding proteins such as transcription factors, in particular transcription activators, for example “zinc finger proteins”, such as Zif 268, proteins having a helix-turn-helix motif, such as the Lac repressor, or proteins having a helix-turn-helix-like motif such as the homeodomain, etc. (Nelson and Cox 2001).

Example of proteins which preferentially bind to single-stranded nucleic acids are single-stranded binding proteins (SSBs). Peptides, such as Ni(II) D/L-Arg-Gly-His, 1,2-dihydro-(3H)-pyrrolo[3,2-e]indole-7-carboxylate (Fang et al. 2004), which preferentially bind to double-stranded nucleic acids can likewise be employed.

Aside from proteins and peptides, it is also possible to use other, low molecular weight, substances. For example, intercalators, semi-intercalators and substances which bind to the nucleic acid surface interact selectively with hybridized or unhybridized nucleic acids, for example with double-stranded or single-stranded nucleic acids. The low molecular weight substances can, for example, particular antibiotics, cytostatic agents or chemotherapeutic agents, such as netropsin, antitumor antibiotic (+)—CC-1065, chromomycins (chromomycin A3), actinomycins, anthracyclines, for example adriamycin, mithramycin, distamycin A and brostallicin (PNU-166196). It is likewise conceivable to use hybrid molecules which are composed of different components, such as S-3-nitro-2-pyridinesulfenylcysteine (Baraldi et al. 2001; Shim et al. 2004).

Other compounds which are suitable are phenanthridines such as ethidium bromide, ethidium homodimer-1, ethidium homodimer-1, hexidium iodide, propidium iodide and dihydroethidium, indoles and imidazoles such as DAPI (4′,6-diamidino-2-phenylindole dihydrochloride) and DIPI (4′,6-(diimidazolin-2-yl)-2-phenylindole dihydrochloride), bisbenzimide dyes such as Hoechst 33258 and 33342, acridines such as acridine orange, acridine homodimer, AC-MA (9-amino-6-chloro-2-methoxy-acridine) and cyanine dyes such as SYBR dyes, e.g. SYBR Green I and SYBR Green II, SYBR Gold, SYBR Safe, PicoGreen, OliGreen, RiboGreen, TO-PRO (1, 3, 5, etc.), PO-PRO (1, 3, 5, etc.), BO-PRO (1, 3, 5, etc.), YO-PRO (1, 3, 5, etc.), cyanine dimers, such as POPO (1, 3, 5, etc.), BOBO (1, 3, 5, etc.), YOYO (1, 3, 5, etc.), TOTO (1, 3, 5, etc.), JOJO (1, 3, 5, etc.), LOLO (1, 3, 5, etc.), SYTOX and SYTO dyes, (7-amino)actinomycin D, hydroxystilbamidine, LDS 751, etc. (Haugland 2002; Vitzthum and Bernhagen 2002) as well as polycationic compounds such as ornithine and spermidine, and also the derivatives, dimers and polymers of the abovementioned compounds.

Modulations in the test system which are based on the nature of the reactants employed, or on their interaction, are described below:

The above-described bases for the change in the activity of the molecular switch in the presence of an analyte make it clear that, when substrates of differing size are used, the steric circumstances which are described are exploited in order to employ different substrate specificities for detecting the analyte. Smaller substrates have better access to the catalyst than do large substrates and are therefore more likely to be transformed. It is only when the analyte-specific conformational change creates adequate access to the catalyst for large substrates as well that these latter are transformed to any significant degree.

In a general manner, it applies that, while the analyte can be a constituent of a substrate, it is preferably not. The presence of an analyte in a sample is determined by adding both a molecular switch which is specific for the analyte and a substrate which is matched to the indicator system. The conversion of the substrate at the catalytic component (preferably an enzyme or a catalytically active nucleic acid) is then the quantitative indicator for the presence of an analyte. The conversion of the substrate is therefore as a rule the auxiliary reaction or secondary reaction which is measured.

In this connection, the respective reactions of small and large substrates can in principle be carried out in parallel or separately from each other. Reacting two or more substrates in parallel is appropriate when the substrates can be detected independently of each other or, in the sense of a competition or inhibition, the reaction of one of the substances is altered such that this process can be detected. This has the advantage that both the original and the analyte-induced conformational states of the molecular switch can be detected.

This is of importance when quantifying analytes since, in this case, several molecular switches, rather than just one molecular switch, are as a rule employed simultaneously in the system. In the ideal case, the sum of the respective relative activities in the system should remain constant. Undesirable side effects are indicated if this is not the case. These side effects may be nonspecific conformational states, such as the destruction or denaturation of the catalyst, or the presence of unknown interfering substances or inhibitors in the sample (matrix effects). Consequently, this internal control can improve the sensitivity, specificity and reproducibility of the system.

It is, for example, possible to conceive of using comparable concentrations of a small substrate and a large substrate, with the affinity of the large substrate being greater than that of the small substrate. The concentrations are preferably to be selected in a range which in each case leads to a 1st order reaction. If, now, the large substrate has a higher affinity than the smaller substrate, the conversion then shifts in the direction of the large substrate, to a degree which is proportional to the difference in affinity and size between the substrates, when an analyte-induced conformational change which improves access to the catalyst takes place. If the analyte-induced conformational change results in poorer access to the catalyst, the system then reacts in the converse manner.

It is likewise possible to conceive of using substrates which are of differing size but of comparable affinities. In this case, the smaller substrate is preferably used in a concentration range which leads to a 1st order reaction. On the other hand, the larger substrate is added in excess such that it is preferably converted in accordance with a 0 order reaction. Under these conditions, the conversion shifts in the direction of the large substrate to a degree which is proportional to the concentration difference between the substrates, or inversely proportional to the concentration of the small substrate, when an analyte-induced conformational change which improves access to the catalyst takes place. If the analyte-induced conformational change results in poorer access to the catalyst the system then reacts in the converse manner. Since both substrates are determined in connection with detecting for increasing sensitivity or for internal control, i.e. negative control versus positive control, the concentration of the small substrate should not become too low. In this case, it is necessary to employ substrate concentrations which are in each case optimally adjusted when combining the aspects of detection and of substrate competition.

It is naturally also possible to conceive, in accordance with the above description, of employing small substrates having low affinities in a concentration range which leads to a 1st order reaction and of using large substrates having high affinities, which substrates are employed in the substrate saturation range, i.e. giving rise to a 0 order reaction.

Instead of a substrate competition, it is likewise possible to conceive of creating inhibition conditions by employing substrate-inhibitor combinations, rather than substrate combinations, in the above-described methods.

If no large substrates are available, it is possible, according to the invention, to prepare large substrates from small substrates by conjugating with substances. It is possible to produce substrates of differing sizes depending on the size of the substance which is used. This provides the possibility of modulating the catalytic activity of the system within given limits. This is at least possible as long as the conjugate formed from substrate and substance can still be converted by the catalyst.

It is naturally also possible to make use, as large substrates, of substrate conjugates or prosthetic groups such as lipoic acid-lysyl residues of enzymes derived from multienzyme complex systems. For example, when dihydrolipoamide dehydrogenase is being used as the catalytic component of the molecular switch, it is also possible to employ, in addition to free lipoic acid and its derivatives as well as formazan dyes, the E-2 components of 2-oxoacid dehydrogenase multienzyme complexes, where appropriate in combination with their E-1 components and corresponding substrates.

If enzymes, catalytically active antibodies and nucleic acids are employed as catalytic components, it may naturally also be appropriate to use substrates, cosubstrates, or inhibitors which are of different but approximately equal sizes. In this case, the conformational change in the catalytic component can also occur in association with a conformational change in the probe. As a consequence of these conformational changes, the substrate specificity of these catalytic components can change without the dimensions of the access to the catalytic component playing a significant role in this connection. It is naturally likewise possible to conceive of conformational changes which effect the substrate specificity in the actual active centre, and change the dimensions of the access to the catalytic component, being augmented.

Measures which can be widely employed in the test system according to the invention and which serve, in particular, to increase both the specificity and the sensitivity of the test system towards a given analyte, are described below by way of example. This is also of particular importance in connection with determining analytes quantitatively.

In order to increase the specificity and sensitivity of the detection of an analyte still further, it is possible to bind more than only one type of molecular switch to one type of analyte. It is, for example, conceivable to employ these two different types of molecular switches which have different enzymatic activities and which in each case bind to different binding sites on the identical analytes. Consequently, these analytes are only detected when the enzymatic activities of all the molecular switches which are bound to this analyte type are altered. An alteration in the enzymatic activity of only one molecular switch would be insufficient. This thereby results in a significant increase in specificity, with this also having a positive effect on the signal/noise ratio and consequently being able to increase sensitivity.

In a preferred embodiment, the enzymatic activities of the different molecular switches which bind to one type of analyte are to be coordinated with each other. Enzymatic activities which permit coupled reactions are to be given particular consideration in this connection. That is to say, the product of one molecular switch constitutes the substrate for a further molecular switch, which then subjects this substrate to further reaction. Since only the end products of the reactions are determined, this determination involves combining the two binding events and consequently permits specific and sensitive detection of the analyte. Preference is given to a product-substrate cycle, as for example in the case of redox cycling, being able to take place within the coupled reactions. This leads to a further increase in sensitivity.

For example, two molecular switches, one possessing lactate dehydrogenase activity and one possessing diaphorase activity, can be employed for detecting a particular nucleic acid sequence. These molecular switches are designed such that their probes recognize different regions of the nucleic acid sequence to be determined. The respective enzyme activities are activated when binding takes place to the homologous regions. If lactate, NAD and a tetrazolium compound are present in the reaction mixture, the tetrazolium compound is only produced to a formazan dye when both molecular switches have become enzymatically active as a result of binding to the analyte, i.e. the nucleic acid sequence. The lactate dehydrogenase activity converts lactate into pyruvate, with the NAD being reduced to NADH. The NADH is then regenerated into NAD in the diaphorase-catalyzed reaction when the tetrazolium salt is reduced to the formazan dye. The NAD is then once again converted into NADH in the manner of a redox cycling, etc. Determining the formazan dye consequently offers the possibility of specifically and sensitively determining the analyte.

Preference is given to the different binding sites for the molecular switches to be located as close as possible to each other. This permits efficient product or substrate transfer between the enzymes. When such spatial factors are considered, it is possible to conceive of also being able to use, in addition to molecular switches, enzymes which are provided with a component exhibiting affinity but which do not alter their enzymatic activity in connection with a binding event. Simply the fact of such an enzyme being in spatial proximity would have an influence on the rate of the overall reaction and have a corresponding effect on the determination.

Consequently, the preferred probes for the molecular switches according to the invention are nucleic acids or nucleic acid derivatives which undergo a conformational change due to contact with, or binding to, an analyte, as a result of which change the access of a substrate to the catalytic component of the molecular switch is either facilitated or impaired. This change in the catalytic activity of the switch, or the change in conversion of the substrate, is as a rule the parameter which is to be measured for qualitatively and quantitatively determining an analyte.

Important aspects and elements of the present invention are summarized below, without the invention being limited to these aspects and elements:

### a) Description of the Preferred Probes According to Generic Terms/Substance Classes

Classification in accordance with the hybridization state:
Hybridized probes
   Intermolecularly hybridized probes
   Intramolecularly hybridized probes
Unhybridized probes

Classification of the probes in accordance with their structure, comprising
Nucleic acid or nucleic acid derivative (backbone, bases) which, where appropriate, comprise at least one coupling component and blocking component but no binding component. These probes are suitable, in particular for detecting nucleic acids or nucleic acid derivatives.
Nucleic acid or nucleic acid derivatives in combination with at least one further binding component (receptors, enzymes, antibodies or their binding domains and also Affibodies, designed repeat proteins, protein scaffolds, aptamers, etc.). At least one coupling component and/or blocking component can also be present where appropriate. These probes are particularly suitable for detecting analytes which are not nucleic acid or nucleic acid derivatives.

### b) Description of the Preferred Catalytic Components According to Generic Terms/Substance Classes

Inorganic compounds:
Inorganic acids and bases, metals, alloys, metal oxides, complexes, transition metal complexes
Example: potassium hexacyanoferrate (transition metal complex)

Organic compounds:
Organic acids and bases, proteins (enzymes, i.e. classical enzymes and also antibodies), nucleic acids possessing enzymatic activity, redox active aromatic compounds and heteroaromatic compounds
Examples: diaphorase, hexokinase, galactose oxidase, etc.

Electrode systems:
Inorganic electrode systems (metals, ceramics)
Organic electrode systems (conductive plastics, compound materials/composites)
Example: gold electrode

### c) Description of the Preferred Combinations of a) and b)

It is naturally in principle possible to conceive of any combination of probe and catalyst.

However, combinations of probes and enzyme catalyst are preferred. In this connection, the use of probes which are constructed in a simple manner is to be preferred, in particular. That is to say that an oligonucleotide is coupled to an enzyme which is preferably a monomer which is stable towards denaturation. Particular preference is to be given to an oligonucleotide which exhibits an intramolecular hybridization which has an influence on the activity of the molecular switch. In this embodiment, preference is given, in particular, to a special version in which the free end of the oligonucleotide is provided with a blocking component.

For example, a diaphorase can be coupled to an oligonucleotide by way of its 5′ end. The coupling can be effected by way of a Schiff's base. In this connection, it is possible to conceive of a 5′ aldehyde-functionalized oligonucleotide having been condensed with an amino group of the diaphorase resulting in the formation of a Schiff's base. Where appropriate, this Schiff's base was also reduced with sodium cyanoborohydride in order to convert the hydrolysis-sensitive Schiff's base into a more stable bond. The oligonucleotide exhibits intramolecular hybridization such that its 3′ end is facing the diaphorase. The 3′ end is functionalized with a biotin group. This leads to an additional reduction in the activity of the molecular switch. An avidin or streptavidin is preferably bound to this biotin group such that the activity of the molecular switch is minimized still further.

### d) Description of the Preferred Analytes According to Generic Terms/Substance Classes

In relation to the invention, it is appropriate to make a division into nucleic acids and their derivatives in contrast to analytes which are not nucleic acids or their derivatives. The latter can be either low molecular weight substances or macromolecules.

In principle, any substance can be the analyte. Very different possibilities for categorizing exist in this connection. At a fundamental level, analytes can be subdivided into atoms and molecules. The substances which are molecules can, for example, be subdivided more or less artificially, in accordance with their molecular size, into low molecular weight substances and higher molecular weight substances, i.e. macromolecules. It is furthermore also possible to subdivide in accordance with functions, such as pharmaceuticals, hormones, metabolites, enzymes, structural proteins and receptors. It is possible to differentiate substance classes in the chemical sense into inorganic and organic substances, with the latter being differentiated in accordance with functional groups, i.e. a classification according to amides, in particular peptides and proteins, acids, in particular carboxylic acids or phosphoric acids, in this case preferably nucleic acids such as ribonucleic acids, deoxyribonucleic acids and derivatives, etc.

### e) Description of the Preferred Combinations of c) and d)

Even if other analytes can be detected, particular preference is given to detecting nucleic acids. For example, HIV-1 can be detected using a molecular switch described in c) when

5′-GCGAGCCTGGGATTAAATAAAATAGTAAGAATGTATAGCGCTCGC-3′ is used as the oligonucleotide. The underlined region corresponds to the sequence which hybridizes with the analyte, i.e. the nucleic acid sequence of HIV-1. The bases in bold are used for the intramolecular hybridization of the probe.

Molecular switches which are provided with a probe which is composed of a nucleic acid or a nucleic acid derivative are preferably used for detecting nucleic acids or nucleic acid derivatives.

Analytes which are not nucleic acids or nucleic acid derivatives are preferably detected using molecular switches which are provided with a probe which contains a nucleic acid or a nucleic acid derivative in combination with at least one binding component (receptors, enzymes, antibodies or their binding domains, and also Affibodies, designed repeat proteins, protein scaffolds, aptamers, etc.).

### f) Description of the Structure and Manipulation of the Preferred Test Systems

The analytical test system based on the molecular switch can be employed flexibly. Basically, the molecular switch makes it possible to record a binding event. In this connection, the nature of the recording depends on the nature of the reaction which takes place.

Since energetic changes occur in connection with virtually all described processes, for example in connection with enzyme reactions, it is in principle possible to employ calorimetry or microcalorimetry using calorimeters containing the reaction vessels which are envisaged for this purpose. Optical measurements can be carried out if spectral properties of the solution change, for example as a result of the involvement of luminescent, in particular fluorescent, and absorbent compounds, in particular in connection with enzyme-catalyzed reactions. In this case, optical measurements comprise luminometry, fluorimetry, photometry, polarimetry, polarometry, etc., using the appropriate appliances and reaction vessels. In principle, it is also possible to detect visually, i.e. by, for example, using test strips, simple cuvettes or microtiter plates, etc. It is likewise possible to conceive of radiometric methods when radionucleotides are used in reactions. In addition to this, it is also possible to employ methods, such as manometry, which record differences in pressure. While this is of interest when osmotic processes are taking place, it is also of interest when gases are formed or consumed, for example when using decarboxylases. Amperometric methods and corresponding equipment, as are used, for example, in polarography, are to be employed in connection with electrochemical processes which use electrodes. This also includes determining potential differences, currents, impedances, etc., and changes in these parameters.

### EXEMPLARY EMBODIMENTS

### 1st Exemplary Embodiment

Glucose 6-phosphate dehydrogenase (G6PDH) is conjugated to an oligonucleotide having the sequence 5′-gtatctagctatgttgatggtg-3′. The 5′-SH-modified oligo-nucleotide is coupled using a heterofunctional, non cleavable, water-soluble crosslinker. The crosslinker sulfo-EMCS is used in accordance with Pierce's instructions (product sheet) (name to be given 2003-2004). This conjugation is preferably effected in the presence of glucose 6-phosphate and/or β-NADH. The abovementioned sequence is used for detecting bacteriophagelambda DNA (λ).

The purified molecular switch, i.e. G6PDH×λ, is then used for detecting bacteriophage DNA as follows. G6PDH×λ is incubated with samples. These samples comprise purified DNA which is firmly denatured beforehand and which is consequently present as single-stranded DNA. The incubation is carried out in 5 to 500 mM tris-HCl, pH 6.5 to 10, and 5 to 500 mM NaCl, but, in particular, in about 50 mM tris-HCl, pH 9, 50 mM NaCl. The temperature is between 4 and 70° C., in particular, however, between 30 and 55° C. and preferably about 35° C. After an incubation of from 0.5 to 60 minutes, but, in particular, of between 2 and 10 minutes, preferably of about 5 minutes, the detection reaction in accordance with Bergmeyer (Bergmeyer 1965) is started by adding magnesium chloride, glucose 6-phosphate and β-NADP. The detection is carried out spectrophotometrically in a quartz cuvette using a spectrophotometer at a wavelength of about 340 nm. The change in absorption is measured over a given time interval and in this way the conversion, or the reaction rate, is determined. When bacteriophage DNA is present, the conversion is proportionally reduced in dependence on the quantity of bacteriophage DNA. The bacteriophage DNA which is bound blocks the access of the substrates to the catalytic centre.

### 2nd Exemplary Embodiment

By way of example, a diaphorase, in particular the Clostridium kluyveri diaphorase, is coupled to an oligonucleotide by way of its 5′ end. The coupling is effected, for example, in the manner described in the 1st exemplary embodiment. The oligonucleotide possesses an intramolecular hybridization such that its 3′ end faces the diaphorase. The 3′ end is functionalized with a biotin group. This results in an additional reduction in the activity of the molecular switch. In one particular embodiment, an avidin or streptavidin is bound to this biotin group so as to minimize the activity of the molecular switch still further.

In addition, this has the advantage of efficiently purifying the molecular switch. The conjugate, composed of diaphorase and oligonucleotide, which is obtained after the coupling reaction is separated off from the excess oligonucleotides by means of gel filtration chromatography. After that, the switch was conjugated with streptavidin. In a further gel filtration chromatography, the diaphorase×oligonucleotide×streptavidin switch is then purified from other substances. The gel filtration chromatographies are in each case carried out in accordance with the customary rules of the technique.

The oligonucleotide

5′-GCGAGCgtatctagctatgttgatggtgGCTCGC-3′ is, for example, used for detecting bacteriophage DNA. The region written in small letters corresponds to the sequence which hybridizes with the analyte, i.e. the nucleic acid sequence of the bacteriophage DNA. The bases in bold are used for the intramolecular hybridization.

The purified molecular switch diaphorase×λ×streptavidin (DLS) is used for detecting bacteriophage DNA as follows. DLS is incubated with samples. The samples comprise purified DNA. An incubation in 5 to 500 mM tris-HCl, pH 6 to 10, in particular, however, in 50 mM tris-HCl, pH 8.6, 50 mM NaCl, is carried out first of all. The temperature is between 4 and 98° C., in particular, however, between 30 and 95° C. and preferably about 80° C. After an incubation of from 0.1 to 20 minutes, in particular, however, of between 2 and 10 minutes, preferably of about 5 minutes, the nucleic acids had been converted into single strands. After that, the temperature was lowered down to from 40 to 70° C., in particular, however, to from 50 to 60° C., preferably, however, to about 55° C. When bacteriophage DNA is present, it hybridizes with the molecular switch probe. If no bacteriophage DNA is present, the intramolecularly hybridized probe is formed once again. The detection reaction is effected, in accordance with Bergmeyer (Bergmeyer 1965), by adding iodonitrotetrazolium chloride (INT), β-NADH and NAD. Other tetrazolium salts, such as neotetrazolium chloride (NT), thiocarbamyl nitro blue tetrazolium chloride (TCNBT), tetra nitro blue tetrazolium chloride (TNBT), nitro blue tetrazolium chloride (NTB), benzothiozolylstyrylphthalhydrozidyltetrazolium chloride (BSPT), WST-1, WST-3, WST-4 and, in particular, cyanoditolyltetrazolium chloride (CTC), are used instead of INT where appropriate. The detection is effected by determining absorption of fluorescence in cuvettes or microtiter plates using appropriate spectrophotometers or fluorimeters at the appropriate wavelengths. The change in the signals is measured at a particular time point or over a given time interval and the conversion or the reaction rate is determined in this way. In the presence of bacteriophage DNA, the conversion is increased proportionally depending on the quantity of bacteriophage DNA. The bacteriophage DNA which is bound removes the blocking of the access to the catalytic centre.

In order to obtain an increase in sensitivity, an NAD/NADH redox cycling is introduced, where appropriate, by adding a dehydrogenase, in particular a lactate dehydrogenase or a formate hydrogenase, and the corresponding substrates lactate and formate, to the reaction (Bergmeyer 1965).

### 3rd Exemplary Embodiment

The 2nd exemplary embodiment is altered in that a hexokinase is used instead of the diaphorase. As a result of the hexokinase undergoing an induced fit when binding the substrate, the effect is particularly large in this case. If no bacteriophage DNA is present, the intramolecularly hybridized probe surprisingly effects the induced fit process such that there is a change in the conversion of the substrate. By contrast, there is less influence on the conformational change giving rise to the induced fit when bacteriophage DNA is present and has bound to the probe.

### 4th Exemplary Embodiment

In one particular embodiment, the hexokinase used in the 3rd exemplary embodiment is employed together with the probe 5′-GCGAGCgtatctagctatgttgatggtgGCTCGC-3′. In this case, the molecular switch consequently has the structure hexokinase-5′-GCGAGCgtatctagctatgttgatggtgGCTCGC-3′-biotin-streptavidin. The following molecular switch is used in addition: glucose 6-phosphate dehydrogenase-5′-GCGAGCctgtacgtgtggcagttgctGCTCGC-3′-biotin-streptavidin. The probe belonging to this switch is also used for detecting bacteriophage DNA but in another sequence region. The experimental conditions which are selected are those used in exemplary embodiment 2. However, in this assay, the bacteriophage DNA is detected by two molecular switches. This unexpectedly increased the specificity of the detection. The overall reaction:

D-glucose+ATP-G6PDH→glucose 6-phosphate+ADP

Glucose 6-phosphate+NADPH+H⁺-G6PDH→Gluconate 6-phosphat+NAD

can only take place when both binding events are successful. Following Bergmeyer (Bergmeyer 1965), D-glucose, ATP and NADPH are used as the substrates. The oxidation of the NADPH is monitored photometrically or fluorimetrically.

This exemplary embodiment demonstrates that it is also possible to carry out a multiplexing. If enzymes which catalyze different reactions which can be detected more or less independently of each other, and probes which detect different analytes, are used, it is then possible to determine different analytes in one and the same assay.

### 5th Exemplary Embodiment

In a further exemplary embodiment, G6PDH is conjugated, as detailed in the 1st exemplary embodiment, to the oligonucleotide aptamer 5′SH-TGGTTGGTGTGGTTGGT-3′ for the purpose of binding human alpha-thrombin (thrombin). The purified molecular switch, i.e. G6PDH×thrombin, is brought into contact with human alpha-thrombin at between 4 and 70° C., preferably at about 25° C., in about 20 mM tris/HCl, pH 7.4, 140 mM NaCl, 5 mM KCl, 1 mM CaCl₂, 1 mM MgCl₂ and 5% (v/v) glycerol. The binding of the human alpha-thrombin to the oligonucleotide aptamer, and the conformational change in the oligonucleotide aptamer which is thereby induced, lead to a change in the activity of the molecular switch, which change was, in a given concentration range, proportional to the concentration of the analyte, i.e. of the human alpha-thrombin. The activity of the molecular switch is carried out, in accordance with the directions given in the 1st exemplary embodiment, by adding the substrates which are described in that embodiment and procedural instructions.

### 6th Exemplary Embodiment

In addition, the abovementioned coupling methods are used to construct a galactose oxidase×5′-GCGAGCgtatctagctatgttgatggtgGCTCGC-3′×biotin/streptavidin switch for detecting bacteriophage DNA. A variety of substrates are employed in the detection reaction, which is carried out in accordance with the descriptions given in Bergmeyer (Bergmeyer 1965) or in Molecular Probes—Fluorescence Microplate Assays (Molecular Probes 1998). The conversion of galactosylated protein is influenced more strongly by the binding of the bacteriophage DNA than is the conversion of galactose. The conversion of the galactose serves as a reference for estimating the overall activity of the system or the change in this activity resulting from the reaction conditions, for example as a result of side reactions and inactivations. The conversion of the galactosylated protein is the actual indicator for the binding of the bacteriophage DNA. Both values are taken into consideration for optimally determining the concentration of the bacteriophage DNA. The difference or the quotient of the two activities is, for example, used for this purpose.

### 7th Exemplary Embodiment

Glucose 6-phosphate dehydrogenase (G6PDH) is conjugated, in accordance with the descriptions given the 1st exemplary embodiment, to an oligonucleotide having the sequence 3′-GCGAGCcatag-5′. However, in this case, the conjugation takes place by way of the 3′ end. In order to generate an intermolecularly hybridized probe, hybridization then takes place with 5′-CGCTCGgtatctagctatgttgatggtg-3′. The hybridization of the intermolecular probe is consequently effected by way of the sequence region which is written in bold. The sequence region which is written in small letters is used for recognizing the bacteriophage DNA. The region which is written in italic is used both for the hybridization of the intermolecular probe and for recognizing the bacteriophage DNA. In particular exemplary embodiments, the oligonucleotide 5′-CGCTCGgtatctagctatgttgatggtg-3′ is conjugated at its 5′ end to biotin and, where appropriate, streptavidin.

Other particular exemplary embodiments are systems in which additional nucleotides, which function as inhibitors, are coupled on at the 5′ end. Other inhibitors which are also used in this case, in addition to the nucleosidemonophosphates, are nucleosidediphosphates and nucleosidetriphosphates, in particular ATP, and also myristic acid, dihydroepiandrosterone and palmitoyl-CoA. In particular exemplary embodiments, it is furthermore possible for β-NADH, α-NADH, β-NADPH, α-NADPH, G6P and gluconate 6-phosphate to be conjugated at the 5′ end.

The purified molecular switch G6PDH×λ is then used for detecting bacteriophage DNA as follows. G6PDH×λ is incubated with samples. The samples comprise purified DNA which has been previously denatured thermally and which is consequently present as single-stranded DNA. The incubation is effected in from 5 to 500 mM tris-HCl, pH 6.5 to 10, and from 5 to 500 mM NaCl, in particular, however, in about 50 mM tris-HCl, pH 9, 50 mM NaCl. The temperature is between 4 and 80° C., in particular, however, between 30 and 60° C. and preferably about 50° C. After an incubation of from 0.5 to 60 minutes, in particular, however, of between 2 and 10 minutes, preferably of about 5 minutes, the detection reaction is started, in accordance with Bergmeyer (Bergmeyer 1965), by adding magnesium chloride, glucose 6-phosphate and β-NADP. The reaction is carried out at a temperature of between 4 and 50° C., in particular, however, between 10 and 40° C., and preferably at about 37° C. The detection is effected as described in the 1st exemplary embodiment.

When bacteriophage DNA is present, the conversion is increased proportionally in dependence on the quantity of bacteriophage DNA. The binding of the noncovalently bound probe component to the bacteriophage DNA now enables the substrate to gain access to the catalytic centre. The choice of the sequence, and of the experimental conditions, prevents any significant reassociation of the probe components and hybridization of the covalently bound probe to bacteriophage DNA, which hybridization can have a negative influence on the enzymatic activity.

### 8th Exemplary Embodiment

Glucose 6-phosphate dehydrogenase (G6PDH) is conjugated to an oligonucleotide having the sequence 5′-gtatctagctatgttgatggtg-3′. The coupling of the 5-SH-modified oligonucleotide is effected using a water-soluble crosslinker which carries at least one of the abovedescribed selective components such as ethidium bromide homodimer-1.

A corresponding switch [G6PDH×selective component×λprobe] is then used for detecting the bacteriophage DNA as follows. The molecular switch is incubated with samples. The samples comprise purified DNA which is denatured thermally beforehand and which is consequently present as single-stranded DNA. The incubation is effected in from 5 to 500 mM tris-HCl, pH 6.5 to 10, and from 5 to 500 mM NaCl, in particular, however, in about 50 mM tris-HCl, pH 9, 50 mM NaCl. The temperature is between 4 and 70° C., in particular, however, between 30 and 55° C., and is preferably about 35° C. After an incubation of from 0.5 to 60 minutes, in particular, however, of between 2 and 10 minutes, preferably of about 5 minutes, the detection reaction is started, in accordance with Bergmeyer (Bergmeyer 1965), by adding magnesium chloride, glucose 6-phosphate and β-NADP. The detection is effected spectrophotometrically in a quartz cuvette using a spectrophotometer at a wavelength of about 340 nm. The change in the absorption is measured over a given time interval and, in this way, the conversion, or the rate of reaction, is determined. When bacteriophage DNA is present, the conversion is reduced proportionally in dependence on the quantity of bacteriophage DNA.

When bacteriophage DNA binds to the probe, there is a dramatic, unexpected conformational change due to the presence of the selective component. This conformational change blocks the access of the substrates to the catalytic centre in an unexpectedly and extremely efficient manner. The reason for this is the affinity of the selective component for double-stranded DNA. The binding of the selective component to the double-stranded DNA leads to a conformational change in the probe relative to the catalytic component, with this change efficiently blocking access to the active centre.

It is naturally also possible, as mentioned above, to employ systems in which the selective component has a higher affinity for unhybridized nucleic acids, for example single-stranded DNA. In this case, the binding processes, conformational changes and activities of the molecular switches change in a corresponding manner in the absence or presence of an analyte. It is naturally possible for the selective components to be combined with the exemplary embodiments which are described in MA 1250.

### REFERENCE LIST

Baraldi, P. G., G. Balboni, et al. (2001). “Design, synthesis, DNA binding, and biological evaluation of water-soluble hybrid molecules containing two pyrazole analogues of the alkylating cyclopropylpyrroloindole (CPI) subunit of the antitumor agent CC-1065 and polypyrrole minor groove binders”. J. Med. Chem. 44(16): 2536-43.
Becker, H. G. O., W. Berger, et al. (1993). Organikum: organisch-chemische Grundpraktikum [Organic chemistry: Basic practical course in organic chemistry]. Berlin, Heidelberg, Johann Ambrosius Barth Leipzig, Edition Deutscher Verlag der Wissenschaften [German science publishers].
Beier, M., F. Reck, et al. (1999). “Chemical etiology of nucleic acid structure: comparing pentopyranosyl-(2′→4′) oligonucleotides with RNA” Science 283(5402): 699-703.
Bergmeyer, H. U. (1965). Methods of Enzymatic Analysis. New York, Academic Press.
Bisswanger, H. (1994). Enzymkinetik: Theorie und Methoden [Enzyme kinetics: Theory and Methods]. Weinheim, New York, Basel, Cambridge, Tokyo, VCH.
Britten, R. J. and D. E. Kohne (1968). “Repeated sequences in DNA. Hundreds of thousands of copies of DNA sequences have been incorporated into the genomes of higher organisms.” Science 161(841): 529-40.
Fang, Y. Y., B. D. Ray, et al. (2004). “Ni(II).Arg-Gly-His-DNA Interactions: Investigation into the Basis for Minor-Groove Binding and Recognition.” J Am Chem Soc 126(17): 5403-12.
Forrer, P., M. T. Stumpp, et al. (2003). “A novel strategy to design binding molecules harnessing the modular nature of repeat proteins”. FEBS Lett 539(1-3): 2-6.
Galau, G. A., R. J. Britten, et al. (1977). “Studies on nucleic acid reassociation kinetics: rate of hybridization of excess RNA with DNA, compared to the rate of DNA renaturation.” Proc. Natl. Acad. Sci. USA 74(3): 1020-3.
Haugland, R. P. (2002). Handbook of Fluorescent Probes and Research Products, Molecular Probes, Inc., Eugene, Oreg.
Hesse, M., H. Meier, et al. (1965). Spektroscopische Methoden in der organischen Chemie [Spectroscopic methods in organic chemistry]. Stuttgart, New York, Thieme.
Hintsche, R. (1999). “Elektrische DNA-Chiptechnologie [Electrical DNA chip technology].” Medizinische Genetik 11: 12-13.
Howley, P. M., M. A. Israel, et al. (1979). “A rapid method for detecting and mapping homology between heterologous DNAs. Evaluation of polyomavirus genomes.” J Biol Chem 254(11): 4876-83.
Kurreck, J., E. Wyszko, et al. (2002). “Design of antisense oligonucleotides stabilized by locked nucleic acids.” Nucleic Acids Res 30(9): 1911-8.
Leitch, I. J. and J. S. Heslop-Harrison (1994). “Detection of digoxigenin-labeled DNA probes hybridized to plant chromosomes in situ.” Methods Mol Biol 28: 177-85.
Meinkoth, J. and G. Wahl (1984). “Hybridization of nucleic acids immobilized on solid supports.” Anal Biochem 138(3): 267-84.
Molecular Probes, I. (1998). Fluorescence Microplate Assays, Molecular Probes Inc.
Mullis, K. B., Kensington, C. A. (1987). Process for amplifying nucleic acid sequences. U.S. Pat. No. 4,683,202.
Nelson, D. and M. Cox (2001). Lehninger Biochemie [Lehninger, Biochemistry]. Heidelberg, New York, etc., Springer.
Nielsen, P. E. and M. Egholm (1999). “An introduction to peptide nucleic acid.” Curr Issues Mol Biol 1(1-2): 89-104.
Pierce, Perbio. (2003-2004) Handbook & Catalog.
Rehm, H. (2002). Der Experimentator Protein-biochemie/Proteomics [The experimentator, protein biochemistry/proteomics]. Berlin, Spektrum Akademische Verlag Heidelberg.
Rosu, R., V. Gabelica, et al. (2002). “Triplex and quadruplex DNA structures studied by electrospray mass spectrometry.” Rapid Commun Mass Spectrom 16(18): 1729-36.
Saiki, R. K., S. Scharf, et al. (1985). “Enzymatic amplification of beta-globin genomic sequences and restriction site analysis for diagnosis of sickle cell anemia.” Science 230(4732): 1350-4.
Shim, Y. H., P. B. Arimondo, et al. (2004). “Relative DNA binding affinity of helix 3 homeodomain analogues, major groove binders, can be rapidly screened by displacement of prebound ethidium bromide. A comparative study.” Org. Biomol. Chem. 2(6): 915-21.
Skerra, A. (2002). “Lipocalins as a scaffold”. Biochim Biophys Acta 1482(1-2): 337-50.
Skerra, A. (2001). “‘Anticalins’: a new class of engineered ligand-binding proteins with antibody-like properties.” J Biotechnol 74(4): 257-75.
Smith, D., B. D. Collins, et al. (2003). “Sensitivity and Specificity of Photoamptamer Probes.” Mol Cell Proteomics 2(1): 11-18.
Smith, M. J., R. J. Britten, et al. (1975). “Studies on nucleic acid reassociation kinetics: reactivity of single-stranded tails in DNA-DNA renaturation.” Proc. Natl. Acad. Sci. USA 72(12): 4805-9.
Stolowitz, M. L., G. Li, et al. (2002). 1,2-Phenylenediboronic acid reagents and complexes. EP 1 264 833 A2.
Torsvik, V., F. L. Daae, et al. (1998). “Novel techniques for analysing microbial diversity in natural and perturbed environments.” J Biotechnol 64(1): 53-62.
Torsvik, V., J. Goksoyr, et al. (1990). “High diversity in DNA of soil bacteria.” Appl Environ Microbiol 56(3): 782-7.
Vitzthum, F. and J. Bernhagen (2002). SYBR Green I: An ultrasensitive fluorescent dye for double-stranded DNA quantification in solution and other applications. Recent Res. Devel. Anal. Biochem. S. G. Pandalai. Kerala, India, Transworld Research Network, 2: 65-93.
Vitzthum, F., H. Bisswanger, et al. (2000). Biotechnologisch relevante Enzyme aus Katzenhaien. Amtsaktenzeichen, Patentanmeldung, DE-10007531.2 [Biotechnologically relevant enzymes from catsharks. Official reference number, patent application, DE-10007531.2].
Zipper, H., C. Buta, K. Lammle, H. Brunner, J. Bernhagen, F. Vitzthum. (2003). “Mechanisms underlying the impact of humic acids on DNA quantification by SYBR Green I and consequences for the analysis of soils and aquatic sediments.” Nucleic Acids Res 31(7): e39.

1. An analytical test system comprising a molecular switch comprising a probe and a catalytic component.

2. The system as claimed in claim 1, wherein the probe is conjugated to the catalytic component either directly or by way of a coupling component.

3. The system as claimed in claim 2, wherein the catalytic activity of the molecular switch is changed when an analyte contacts the probe.

4. The system as claimed in claim 3, wherein the change in the catalytic activity of the molecular switch is due to a conformational change in the probe which is elicited by the analyte.

5. The system as claimed in claim 1, wherein the probe or a constituent thereof is a nucleic acid or a nucleic acid derivative.

6. The system as claimed in claim 5, wherein the nucleic acid is a ribonucleic acid, a deoxyribonucleic acid, a peptide nucleic acid, or a locked nucleic acid.

7. The system as claimed in claim 5, wherein the nucleic acid or the nucleic acid derivative is present in hybridized form.

8. The system as claimed in claim 1, wherein the probe or a constituent thereof is an oligonucleotide.

9. The system as claimed in claim 8, wherein the oligonucleotide exhibits an intramolecular hybridization.

10. The system as claimed in claim 1, wherein the catalytic component or a constituent thereof is an enzyme, an antibody, a catalytically active nucleic acids or a catalytically active nucleic acid derivative.

11. The system as claimed in claim 10, wherein the catalytically active nucleic acid or the catalytically active nucleic acid derivative is a ribonucleic acid, a deoxyribonucleic acid, a peptide nucleic acid, or a locked nucleic acid.

12. The system as claimed in claim 10, wherein the catalytic component or a constituent thereof is an enzyme.

13. A method for determining the presence or concentration of an analyte in a sample, comprising contacting a molecular switch with a sample and measuring the activity of the molecular switch to thereby determine the presence or concentration of the analyte.

14.-15. (canceled)

16. The method of claim 13, wherein the analyte is a nucleic acid or a nucleic acid derivative.

17. A molecular switch comprising a probe and a catalytic component.
